SEQUENCE LISTING (19 sequences)

SEQ ID NO: 1 - DNA, 23 bp, Paralichthys olivaceus
  source 1..23: mol_type = genomic DNA
cagtgcgtccttcggagactcgg

SEQ ID NO: 2 - DNA, 23 bp, Takifugu rubripes
  source 1..23: mol_type = genomic DNA
gtagatggaacagcacacagtgg

SEQ ID NO: 3 - DNA, 19 bp, Takifugu rubripes
  source 1..19: mol_type = genomic DNA
gtagatggaacagcagtgg

SEQ ID NO: 4 - DNA, 23 bp, Stephanolepis cirrhifer
  source 1..23: mol_type = genomic DNA
ccctcacgcagttcctgaacgcg

SEQ ID NO: 5 - DNA, 23 bp, Plecoglossus altivelis
  source 1..23: mol_type = genomic DNA
aaagtgtccaatcaggaaggagg

SEQ ID NO: 6 - DNA, 23 bp, Scomber japonicus
  source 1..23: mol_type = genomic DNA
ttctggtctgctgagatatgagg

SEQ ID NO: 7 - DNA, 23 bp, Oncorhynchus mykiss
  source 1..23: mol_type = genomic DNA
ccctgctctgcctcacctgtgga

SEQ ID NO: 8 - DNA, 23 bp, Oncorhynchus mykiss
  source 1..23: mol_type = genomic DNA
agggagagttgattctgagctgg

SEQ ID NO: 9 - DNA, 23 bp, Oreochromis niloticus
  source 1..23: mol_type = genomic DNA
ctctctgcgttcaggtgccgtgg

SEQ ID NO: 10 - DNA, 23 bp, Oreochromis niloticus
  source 1..23: mol_type = genomic DNA
ccttcagcccaccttggtgtgga

SEQ ID NO: 11 - DNA, 23 bp, Epinephelus akaara
  source 1..23: mol_type = genomic DNA
tccactcctgagtgcactgcagg

SEQ ID NO: 12 - DNA, 23 bp, Gnathopogon caerulescens
  source 1..23: mol_type = genomic DNA
gactgaatggctctggatactgg

SEQ ID NO: 13 - DNA, 23 bp, Gnathopogon caerulescens
  source 1..23: mol_type = genomic DNA
aagattaagcaccaggcctcagg

SEQ ID NO: 14 - DNA, 23 bp, Gnathopogon caerulescens
  source 1..23: mol_type = genomic DNA
actcggcttcacctgaggcctgg

SEQ ID NO: 15 - DNA, 1516 bp, Stephanolepis cirrhifer
  misc_feature 536..659: note = n is a, c, g, or t
  source 1..1516: mol_type = genomic DNA
tacgcccgccgcatgcgtgtcaaaggtcaaagccgtcatgtgacgaggctgttggtccctcacgcagttcctgaacgcggcccggacttctggaggattctccaagaccatccgaacagacctcagaccaaagtcaccctgttgttcaaggaagtcgacacgtcaggtctctcgtactgtgtggatggttttctaatccagcaccggagctccagtggctctgtgacaaccgagcggatcgagccggccgcctccttcagcttcgactggaaccagaggcccgagaatgtgactgtgatggcctacaacagtttggggaactccagcaacaacagcttcatggtgctggacagacagcccaaacgtcgctgcgtgcgctccttcagcgtgcaggtggtcaacccatcctgcgtctccctgacgtggacgctgtttgagaactgctccacccccgacttcatggtggtccagtggtcctcgcggaggcaccagggctccgcccaccaccgcaggctgcagggcggacacacctgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgaaagaggagaagctgccgtctaccagatgctgctgatcgtcttcttcctctctgtcgtcctgtttgtcaccctcatcatctcgcagcaaaaaatgaagaagtttgtgtggaaggaggtgcccgaccccagcaagtgctcctgggccaaaggagtggacttcagaagggtggaccctcttgaccacatgttccgtcctgccgacgatctcccagccaggccgctgctgctgctgccgcttgaaaacatctccaaattggtcgtcatggacaagatagacctgtcagtgctgaacgtagacgtggtccaagaccccgcagatccagtcgactcggaggccgactgccctcagccacttcctgcttcagagcctccattcgtcctcgacctggacgctttgaccagcttcaaaccagaacaggaagacccggcgaggaaggagaactcggcccagtcttttgtcacctactccactgtgctgctgcaggatccgcagcagcagcaccagcagcagcactacaaagacggcagcagcggcagtagcagcagcagcgacgaaggaaacttctcggccaacaactcggacatctcagggtcgttccagggcggcttgtgggagctggacgacccgcggcgctcctgctccttcaactccgtggagcagctgtcggaggcgtcggagcaggaggacgagagccgagtcagaggcgagaaggacttgttctacctgggggtggaggggtaccctggggacgagagcgaggacgaggaggagaaggatgagacggacaccgagctgctgaagagtgtcccgggcagggaggaacgtcccgccgagtcgcaccctctgctggcgg

SEQ ID NO: 16 - DNA, 3621 bp, Plecoglossus altivelis
  source 1..3621: mol_type = genomic DNA
atgctggccttcctggtactcatcctcttcctctaccatggaactgtgtgtgtggagccagagagacacccacctgcaagggaggaagacctcccatggcagtcccatctttgctgtgacaccctgccccccctgccccccctgcccccccggctccccaggcccccccgtcccccccagcctgggagatccagagaggagctctctgattcaaagaacgtccacagctcctgcatctacttcaactcctccagcatggcatctcctccaccccccacagagagtcaagtgtgtttggatatccagtgtgtaattgacgcgagcggagagaaagtaatttgccatctgaaacctctcgatgattcttccaactccaccagcggcttcacagtaatcaccctgcagcgctctgaacgtggcgaggagagagagcgggaggaggtcgcgaacgccagcgacaccgtcggctgcgcggggacgccgcccttccggtgctccgtcgccgtcggcgccgtcgtcgcggcaaccgtgaacgtgtcgggagccgtggctcccccggtgctgctccgccttctacccgttaacggtaaaccagacccaccagtgaacgttacgcacatcctcaccccagacggagacctcctactgagttggagccccgcccccctcagctccgccccccccggctccgcctacctaagccccgccccgctccgctacgaggtccgctatgcccccagcgcctcccctgctgtttggaaggccccagctccgccccctcgcccttcctccttcctggagcgactggagccaggcttacaacttcctgttggacttgagaagaggaacaacaacaagatgaaggtccacccccccctgcccccccgtccccctgccctcagcccccctgccccccgcccctcagccctcctgccccctgccctcagcccccctgcctccccgcccctcagcccccgccccagcccccctggccctcattacgccagctacttcccagagagggtggcggtccgacgcggggaagacgtgagcctccgctgcttgttcaacgaccctgctgccaagtccagcacctccgtgtggttcctgaactggcaccctgtggaggccaccgtccaccaggtggtagaggtcagccccagggtcagccaggtcagccccagggtcagccaggtcggcaccagggtcagccaggtcagccccagggtcagccaggtcggccccagggtcagccagatcaccctgcggccccctgaggaacgactgcacagcacgctgcagtgctaccagagcagcaacactcgctactccttcaaggttccctacgctgacgtctacgtccacggagcgctcatcgatatccgctgccagaccaacggggtcatggacgccatgacctgcaggtggaacaacagccacagactggaggagctcaccttcttctacaggtcggaagctgtgacctgtgacatcatggaggaggaaggagaggaggaggaggggagcgtggaagagggtcgcagtgagatcaacgggacaaagtgtccaatcaggaaggaggaagggagtgggaggagctgtgttttccagccgctcagactgacgtactgctacaagctgtggctgcagatccagtcagaggacggacccgtgaggtcacatcctgtctacgtcacgcccaccgaaaatgtgaagcctaaccctccagcccaggtgcaggctcatgtctctcccagcagaattctgaagcttgtctggcagcctcctagactgcccttgtacgagctccagtaccaggtccgatacctccccgctgcaggtcgggccctcccagactggcaggaagtgggtccagtgagcgagccaagagcggagatccctgtacctgacgtgtgtggcgtgtacctggtccaggtgcgctgtgtacccaccaatgggactggccactggagcgactggagcaagcctgtctactccacaacccccaaccgcagtgctccccatcatggtccagacttctggaggattctggaggaagattcatccttgaacatcacctacgttactctgctcttcaagcatgtctctatggagccctcctcctgtgtagagggccttgcagttcagcaccaggtctctgggggcgctgtggtcacggagctgatcggcctagttccttcttacacgtttgaatggaaggaggaagtacacacggtgacggtgaaggcgcagaatcgcttgggatcgtcaggtcacaacgtcaacatgaccttggaccgacacgtcaaacgtccgtgtttactctggttcagagcgtccctggtcaacagcagctgtgtggagctatcctgggctctgctgccccagaacccgaccccctggtccctggtgctgcagtggtcaggagccaccagggggcgccggccagcccacgacccgacctcggggaccagcctgaagtgggccagggtgccgttcactgaccgcgttttctacctgcacggagaattctacggctctgaagagtatgagttcatcttgtaccctatatttgcagacagggaaggcgagccaatgcatgctaaagccagcagagggccccctgctgactacatgttgctgattatcatcaccttcctagccatcgtcctcatcatcactctcatgttgttccagaaccacatgaagaagctgatgtggagaaaggttcctaaccccaaccgctgctcctgggcgcaaggaagagacttccggaaggcagagaccatggaccagctgttcagacacccagacggtttccctgcctggcctctcctcctggtgtctgagaccatctctgaggtggtgatcatggagaagagcagccttccctctcctgctctctctccagctccggaccccgtccctgggcaaggccctcccagaggcctccctggccctcgcccctcctccacccccagcgaaggctctggctctgggttccccaggacatcagagacctcggcccagtcgtcggtcgcgtacgctactgttctcctctccaacaagccaagcctcctccgcaagcaagatggaggtagcagctccagcgacgaggggaacttctccgccaacaattcggacatttctggctccttccctgaggggttgtgggggctggacaccctgcggaccggagggcctgacaccagacattcacgctctttcaactccgtggaggagttctccgaaacatcggagcaggaggacgaggcgttggggggggagggagagggggagggagagggggagggggaggagaagagcctgtactatctagatatgggctgtccgcaaagggaggaggaggaggaagaggaggaggaggaggaggaggaggaggggaggttggaagaaaggaaaacactgcttctccaggaacgggaggcgttgtgggcggagcctaactctccgcttgggcggggcgaccccaggttcgagtgcagggaggcggagtcaggtgacatggttggaggcgtggctctttacctgcctcagtttaggactggccccggcagacctggtacagagggaaggccagacagcgccctccagctgtga

SEQ ID NO: 17 - DNA, 3450 bp, Scomber japonicus
  source 1..3450: mol_type = genomic DNA
atgaacacaacaatggtttggtctgtaatgttgtcagtcctgatgcatattttcctggtgtcccatggtgccctgtgtttggagcccgaggaggttgcctctcttcaggcaggtgctctgaacctcccatggcaggatgagctgtgctgtgattcaccgtcagcccacctcactgtgaaggtggacggtgtgcacgcactggagacaaaccactctgaatcaagcctcccacactatgctcactgcaacttcaggagcctgacagccacatcacatcctcacaagccctctggtggcacctgtttggacatgctgtgcagaattgatgaaaagtgggagagtttgacttgtgatcttcagtctcacgacccgccttcagacacaatgaatactgctcttatggcaattagcttacagcatctgctctccaagaaaaagggctcagaggtgaattatgggaatgacgcatcttataaccctgtcgtctgtgaagcagaggattccttcatgtgctcactcccacttgacactacgacaagctttgttaccatggtgactgtcagcatcgctgaagctgtagctccgccaattctgctcagagttcctgcccgacctgtgaaacccagtcctccggtcaacctttcacatatgcagactattgaggcagaactgattttacactgggacgacccagaagacattgattctggtctgctgagatatgaggtccgatactcttctaacaccaatcatccagcctggcaggtggtttctgtacccgcagagcccaggatgcttctggatttgaagcccagactgaactacactatccaggttcgctccggcttggatgaacccccactgtggagtgactggagtgaaccctaccacatttacctggacacggtgagctacatccctgagaaagtggtggcgcgcccgggagagaacgtaacagtttattgtgtattcaacgaccacagcatcaatgccagcacggccatgtggatgctcaacttccaacagccgcttcatcgcagccagtaccacccagtcaaccagtgggtcagccagatcacagtgcgcccttcagagactcgaatgtatgacctgctgcagtgcacgcaggagtggaccatcccctacagccagatctatgtagaaggagcatccattgatataaaatgtgaaaccaacggtgatattgatgctatggactgcagctggaagaacacacagtggactaaacccaaattcctatccaggtgggctgatctgccatgtgatgtaatggaggaaagggagagagcaggtgagagtgtgggagagatgggacctgtttgcctctctcagaaatcctgcaccatccagcccctgaggatgaactgctacaagttgtggctggaggtgccatcccgtctgggccctatctggtccaaacctgtctacctgtcacccatagatcatgtgaaaccacacacaccgactaatgtgagggcagtgagccggagcagcggggtcctgacgatcacatgggaaccaccttctctgccagttgaggggctccagtgtcagtttcgtctccactcactgtccactgtcagagctcagccagagtggaaggttcataatccagtgcgggtgcaatgggcggaggttgcggttccagatatgtgccgtgtgtatgtggtgcaagtgcgctgcatgcacaccaacggcaccggctactggagcgactggagtgactccatttactctacgccacaaaatagcagagctcctgatcgtggccctgatttctggagagttctacaggatgatccatacagaaaccaaactaatgttactctgctatttcagcctctcctgacatcagggcactcctactgtgtggatggatttatagtccagcaccagtcctcaagtggcactgtgataagggagcggatcgagctggtgtcctcctacagctttgagtggaaccaggaggtccaaactgtcaccgtggaagcctacaatagtctggggagctcggctaacaacatcaatatgacactggggagacagcccaaacgccgctctgtgcgtgcgttccgtgcgctggttatcaacagcacctgtgtgtctctgtcctggagcctgctagacaacagctctgtccctctattcatggtggtccagtggttgccacagaggcaacaggaccctcatcatcacaaaggcctgagtggagaaccatgggtcagagtgccttacactgatcgtcctgtctatttaagaggtgatttctttggctctgaggagtatgggttctatctgtatcctgtgtttgttgaaggagaaggggagccagcatacactacagccaccagaggagaccctgcagcctacatgatgctgatgatcatctccttcctctccatcgtcctgtttgtcaccctggtcctctcccaaaaccagatgaaaaagtttatgtggaaggatgtgcccaacccaaacaaatgctcctgggctaaaggactggacttcaaaaaggctgacacgttcgaccaccttttccaaccgccagagggcctgccagcccggccactgctgctcccctctgagaacatttccaaagtcgttattatggaaaagactgacctctcagcgctgaccccgcttgtgtccctaaatcccgacacagcaactgccttagctatctccattccttcagggtttaactctgaggttgaccaaacccgatccatggacagtgaagcgcttctgggtggagctccctccttaacccttgatatggatgctttaaccgcctcaagcctgaggacagatgagttacagcttgtcgacctcccaagaacagatcagctgccaggcaccaataacaacagtgcacagtcctcagtcacttatgccactgtgctgctcattaatccaaagcaggagcagcagcccattcatctccactacaaggatggtagtggcagcagctccagtgatgagggcaatttctctgctaacaactcagacatttcaggatctttccccggtggcctgtgggagctggagagctgccgaggtggggagatggatgacccgcgacgctcctgctcctataactccgtggaggagctttctgaaacatctgagcaagaagatgaggaggaggtgagagaagggaaacacctgtattatctcggtatggactatccagcggaggatgaggagagtgacgaagaagaggagcagagagaggaagatacaaaaactgagctgctcacaagtgttgttttgaacagagatgcctgctctgtggagtcacacccattgcttggccctgtggactcgagtgataacagtgagatggattcagcatcaacacgtggcttttccctgctgtacatgcctcagttcagaactgcaccatgcacgaggcaactcacagattaa

SEQ ID NO: 18 - DNA, 3147 bp, Epinephelus akaara
  source 1..3147: mol_type = genomic DNA
atggactcggacaaaagaagagccggacacaaagccaggggaaacttccaggagcgtttgagattcaccccagacaaagcccagccaggccatgctcacgctatgactactacaatggttcggtctgttatactgacagccctgatgcacttgttcctggtaccccatggtgctcagtgtttggagcctgaaaacggagcctctgaccattcaggtgccctggacctcccgtggcaggatgagctgtgctgtgactcaccttcagcccacctcactctggagggagacgacatgcacgcaccggagacaaaccgctctgaatcaaacctcccacaacatcccctctgcagcttcaggggctcgacaagcgaatcacatccacgtgagccctctggcgtgaaaccaagtcctccagtcaacctgttacataatcagaccattgaggcagacctgattttacaatgggacgacccgtcagactctgataccagtccactgagatacgaagtccgatactcttctaacaccactcatccacagtggcaggtggtgtctgcacctggagagcccaggttgtctctggagctgaagcccagactgaactacaccatccaggttcgctgctccggcctggaaaacccgccagtgtggagcgactggagcgaaccttatcacatctacctggacacggtgagctacatccctgagaaagtggtggcacggccgggggacaacgtcacggtgtattgtgtgttcaatgaccacagcatcaacgccagcacggccatgtggatgcgcaacttccaacagcggcttcatagcagccagtatcacccagttaaccagtgggtcagccagatcacagtgcgtccttcagagtctcggatgtacgacctcctgcagtgcactcaggagtggaccatcccgtacagccagatctatgtacaaggagcttccatagatattacatgcgaaaccaacggtgacatcgacgcgatgacctgcagctggaagagcacacagtggacaagaatcaaattcagatccaggtgggctgacctgcagtgcgacgcgatggaggaaagggagcgagcgggggagaaagtgggggagttggggccctcgtgcctgcaggtccgatccaagcagaaaacctgcaccatccagcctctcaggatgaactgctacaagctgtggctggaggtcccgtcccgactgggccccatcaggtctaaacccatctacctgtcccccatcgatcatgtgaaaccccactcacccactaatgtgaaggcagtgagcagaagcagcggggtcctgatgatctgtgggagcctccgctctgccagtcgaagggctccagtgtcagtttcggtaccactcaccctccgctgtgagagcccagccggagtggaagaggatgttctgacatggactcagctgatccagagtccagtgcgggtgccatgggcggaggtcttagtgccggacatgtgccgggtgtatgttgtacaagtacgctgcatgcacaccaacggcaccggccattggagcgaatggagcgattcggtctactccacacctcagaacagcagagctcctgagcgaggccccgatttctggagagtccttcaagatgatccgtacagaaaccagacgaatgtcactttgctgttcgagcagcagcatctccaactatcaggacggtcttactgcatagatggatttatagtgcagtaccaggcgttgagtggctctgtgacgagggagcagatcgagctggcgtcctccttcagctttgagtggaaccaggtgccccaaactgtgacggtggaggcctacaataatctggggagctccgccaacaacttcaacatgacgctggagagacagcctaaacgccgcagtgtgcgctcgctcagcgtgttggttatcaacagcacctgtgtgactctgtcctggactctgctggacaacaactctgtgcctctgttcatggtggtccagtggtctccacataagcaacaggactcagatcatcagaaaggccggattggagaaacatgggccagactgccctacaccgaccatcccatctacctgaaaggtgatttctttggctctgaggagtgtggcttctacttgtaccctgtgtttgctgatggagaaggggagccggtgtatgctatagcttccagaggagaccctgcagcctacatgatgctgatgatcatctctttcctctccatcgtcctgtttgtcaccctgatcctcacccaaaaccaaatgaaaaggtttgtgtggaaggatgtgcccaaccccaacaagtgctcctgggccaaaggactagacttaaaaaaggctgacaactttgaccacctgtttcaacctgcagagagcctgtcagcctggccgctgctcctgccagctgagaacatttcaaaagtcgtcatagtggatgaagctgatctctcagctctgactacggctttaatccaagtcccacttgtgtccctgactcctgacccagccactgctttatctatctcccttcctccagggtatgactcggaggtcgaccaagcccagcccatggagagtgaggtgcttgtcagtggagctccatcccagctcatggagcagccgccaattcatctccactacaaagatggcagtggcagcagctccagtgacgagggcaatttctctgccaacaactcagacatttctggatctttccccggcggcctgtgggagctggagagctgccgtggtggagagatagacgaccctcggcgctcctgctcctacaactctgtggaggagctttctgaaacatcagagcaagaggatgaggaagaggaggcgagagaagagaaggacttatattatctaggaatggactacccagcggaggatgaggagagcgaggaagacaaggagcagatagaagaagagacagatattgagctacttaaaaatgtggttttgagcagggaggactgctctgtggagttgcaccctttgctcagccctgaggactcgagcgaagttgctgttagcgtcaacacgtggtttttcctcgctgtacctgcctcagttcagaactgctccatgcacgaggaaactcacagctcaaccgcatga

SEQ ID NO: 19 - DNA, 3249 bp, Gnathopogon caerulescens
  source 1..3249: mol_type = genomic DNA
atgatgtatttgtttatcttgctttcgcttcttgcgaatttcattaccgtttcacaaggtcttgctgctctgaatccttcggatggcaggggagtttatgaagacctgaagtggaagtctctgctatgttgtgagctgccctttgctcagacagttgacagtggtctttcagaacaccgaccagtagaaccgtgtcagctgctaaatgttacaaaaccggaatcctctagtaaatcacttgccttttctggaaactgtttggacatcctgtgctggcttgaaggtgaacggacaaatttgatttgtaatgcaaagagccacggagaagcagccactgccagtctgttcactgttagtcctcaacaattagttctacagatggatatactttcagaaaaaaaccatagtgctcagtgtgctggggaagatactaccacgtgctccgtttctcttcatggcaatgatgcaactgtctcgttgaccatcagtatatctgtgaatgggactactgcactgtcaccggagatgcaaatcagcacatatcatctaagaagaccagacccacctgtcaatctgcattacaatgtgacgacagaagacgaagtcattttcagatggagtagcacacaacctgacagtaatgacataaactatgagatccgatactcttccaattcctcgcttcagcagtgggaggtggtgaaggtcagaggtcattcatgggtacctctgaatgagctcagttttgggatcagatacacagtccaagtgcgctgccaaaactacttccattactggagtgaatggagccaacctttctatacactagacgtcagttacatccctgctgaagtctccacaacgcctgggtcagaggtaacggtttatgccgtcttccacaaccgcagctggagtgcgagcaaagctgtgtggatgctaaacgggcatgagaagattccggaaagccagtacagggtaatcaacgagcaagttcgtgctgtcactataagggtagatgaacccaggtttgattctctgatgtgctgccacttgggaggagagcgggtcaaatgccccatcgcctatggcaaaatatacaccgaagggagttttaatgcagatattacctgcgagagcaagatttcagaggtggacaccactctggattgtgagtggaataaaagcgcttgggcactggtcagactcctttacagacaatatatatcaatatgtgaaaccatatcaaagatgcagggcattgaagaagctgaagaaaacatgcctttggtaaaggagtaccaaagtggagcaggagactacagaaaatactctttaagcaaccttagcctgtactcttgttacgaaatctggttggaagtggaaggagggcgtggcaaagtgagatcatttcctgtctatgtttcacccattgaccatgtgaaaccatctcctccctcagtccttgaagcgatcactttgccgaacaaaaccttaagcgtaaagtggaaatatcccgatttacttgtatacaaaatgcagtatgagctgcgctttgtggctttgcgtgggatggcaaatacgcaatggaaggtcatcggtcctctattggaagcacaggcagagattccgcttgaagattcctgtgttcagtttaatgtagacgttcgctgtaaaagactgaatggctctggatactggagcaaatggagcgggagtcacacttccattgtttacaacaggaaagctcctgaaattggaccagacttctggcgcattatacaagaggatcctgttaggagcgtgacaaatgtcacactgatctttaagcagcctatcctagcaggagatccatatagttgtgtggagggtctcaagattaagcaccaggcctcaggtgaagccgagtggtcaaaagaaacggccttgattcagtttcactcattccagtggaagaaggaagcacacacagtcaccgtaatgtctcgcaacgctttgggcttctctacacggaatagcaacatgacgctgttacatcaacccaaacgacgatgtgtacggtcattcagcgcagttgcaaatgcgagctgtgtgcatctctcctggagcctgttacctgatcaactggtgcctcaatcgtttgtcatcgagtggttagacctgaacaaagatcctgaacaggacgggtctctaacggagaggctacagtgggtcagagttcattccgcatccagggatatctctctttgccgtcgtttttatggctcggaggagttcacattgtatccagtgtttgtggatggtgagggggagccggttcgatacacagttactaggagcgactctgctgcctacatactgctactgatcattgcgttcctgtctgtggtgctgtttgtcacactcatgatgtcacaaaaccagatgaaaaaacttgtgtggaaggatgttccaaatcccaacaactgctcctgggccaaaggaatggacttcaggcagattgacaccatggagaacctgttccctcactcagaaggtctcacggcctgtccactgctgctggtctccgagagcatctgcgaggtggaaatcgagaaatgtcatcctctcaaccttgaacatggaaaagacaatgaagtactcctttataactcggaagacaagacaaccaccgactctgcccgtctgggagactcctctgaacctttatcactggaggcttccactgctgctgctaccccagaaacatcaggccagtcctcggtgacatactcgaccgtcctcctcgccgatcagcccgctctccttcgaaagcagcaggagagtctgagcagctccagcgacgaaggcaacttctctgctaacaactcagacatttccggctccttccctggaggactctgggacctggagaaccacgtgtgctctgacagcaccaatcctcgccattccagctcctacaactccgtggaagagttctcggaaacatcagaacaagattacgaagcatcggaaagcactggtgtagccaaagacctctactaccttgatatgaatgaagaggagaagcaggacaaagaagaggatgaggaagctcaggatggacaggataagaatgaaatggttatgagggtggatgccaggcctcttctggaaggcaaaaattccacagacgtcgattccaataatgtatctcacagcgttccgctttatctgcctcagtttcgaactgaatgcatcaatccaccatga

(19) United States
(12) Patent Application Publication
(10) Pub. No.: US 2025/0324954 A1
(43) Pub. Date: Oct. 23, 2025

(54) METHOD FOR PROMOTING SEXUAL MATURATION OF FISH

(71) Applicant: REGIONAL FISH INSTITUTE, LTD., Kyoto-shi, Kyoto (JP)

(72) Inventor: Kenta KISHIMOTO, Kyoto (JP)

(73) Assignee: REGIONAL FISH INSTITUTE, LTD., Kyoto-shi, Kyoto (JP)

(21) Appl. No.: 18/865,819

(22) PCT Filed: Jun. 16, 2023
(86) PCT No.: PCT/JP2023/022471

§ 371 Date: Nov. 14, 2024

(30) Foreign Application Priority Data
Jun. 17, 2022 (JP) 2022-098322

(51) Int. Cl. A01K 67/0276; C12N 9/22; C12N 15/11; C12N 15/113

(52) U.S. Cl. CPC A01K 67/0276; C12N 9/226; C12N 15/111; C12N 15/1138; A01K 2207/05; A01K 2217/054; A01K 2227/40; C12N 2310/14; C12N 2310/20

(57) ABSTRACT

The present invention provides a method for promoting sexual maturation of fish, the method including a step of suppressing functional expression of at least one of a leptin receptor and a leptin of the fish. An object of the present invention is to provide the method for promoting the sexual maturation of the fish in order to obtain individuals of fish capable of ovulation or spermiation at one year of age at a higher rate than in nature or under normal aquaculture conditions.

### TECHNICAL FIELD

The present description discloses a method for promoting sexual maturation of fish.

### BACKGROUND ART

Fish are supplied to the market either as natural marine resources or through aquaculture. Among fish, for example, it has been reported that female individuals of bastard halibut (Paralichthys olivaceus) require two or more years to reach sexual maturity as adult fish (Non Patent Literature 1 and 2).

### CITATION LIST

### Non Patent Literature

[Non Patent Literature 1] Ozawa, Takakazu et al. Nippon Suisan Gakkaishi, 1996, 62(5): 733-739
[Non Patent Literature 2] Yamamoto, Masayuki et al. Bulletin of the Kagawa Prefectural Fisheries Experimental Station, 2010, 11: 1-15

### SUMMARY OF INVENTION

### Technical Problem

In the case where fish can be brought to sexual maturity earlier under aquaculture, fish production under the aquaculture can be increased, and aquaculture costs can be reduced. It can also shorten a generation period, which is a rate-limiting step in breeding, and thus reduce time and development costs of producing high-quality breed varieties.

However, according to Non Patent Literature 1, very few female individuals of bastard halibut (Paralichthys olivaceus) can ovulate at one year of age in nature. In Non Patent Literature 2, it is also described that most individuals of bastard halibut (Paralichthys olivaceus) of both sexes are considered sexually mature at two or more years of age, and that the minimum age of maturity is two to three years of age.

An object of the present invention is to provide a method for promoting sexual maturity of fish in order to obtain individuals of fish capable of ovulation or spermiation at one year of age at a higher rate than in nature or under normal aquaculture conditions.

### Solution to Problem

The present invention includes the following aspects.

### Item 1.

A method for promoting sexual maturation of fish, the method including a step of suppressing functional expression of at least one of a leptin receptor and a leptin of the fish.

### Item 2.

The method according to item 1, in which the functional expression is suppressed by introducing a loss-of-function mutation in a gene of at least one of the leptin receptor and the leptin.

### Item 3.

The method according to item 2, in which the loss-of-function mutation is introduced by at least one genome editing system selected from a clustered regularly interspaced short palindromic repeats/CRISPR associated protein 9 (CRISPR/Cas9) system, a CompoZr Zinc-finger nuclease (ZFN) system, and a TAL effector nuclease (TALEN) system.

### Item 4.

The method according to item 1, in which the functional expression is suppressed by suppressing expression of a gene of at least one of the leptin receptor and the leptin.

### Item 5.

The method according to item 4, in which the expression of the gene is suppressed by at least one type selected from a group including at least one type of an RNA molecule selected from a group including a SiRNA, a shRNA, and a miRNA targeting mRNA of at least one of the leptin receptor and the leptin, or a vector capable of expressing such an RNA molecule.

### Item 6.

The method according to item 1, in which the functional expression is suppressed by suppressing a function of a protein of at least one of the leptin receptor and the leptin.

### Item 7.

The method according to item 6, in which the function of the protein is suppressed by at least one type selected from a group including antibodies that specifically bind to at least one of the leptin receptor and the leptin to suppress functional expression of a binding target by such binding.

### Item 8.

The method according to item 1, in which the fish is a female individual.

### Advantageous Effects of Invention

It is possible to obtain fish individuals having an ability of ovulation, spawning, or spermiation at one year of age at a higher rate than in nature or under normal aquaculture conditions.

### BRIEF DESCRIPTION OF DRAWINGS

FIG. 1 shows a temperature condition during rearing.

FIG. 2A shows presence or absence of ovulation in female individuals of bastard halibut (Paralichthys olivaceus) deficient in leptin receptor genes and weight changes at ages of 5.1, 10.1, 12.4, 15.8, 16.1, and 16.8 months.

FIG. 2B shows presence or absence of spermiation in male individuals of bastard halibut (Paralichthys olivaceus) deficient in leptin receptor genes and weight changes at ages of 5.1, 10.1, 12.4, 15.8, 16.1, and 16.8 months.

FIGS. 3A and 3B each show the weight changes in the female individuals of bastard halibut (Paralichthys olivaceus) deficient in leptin receptor genes, in which FIG. 3A shows a group in which the ovulation has been observed, and FIG. 3B shows a group in which the ovulation has not been observed.

FIG. 4 shows the weight changes in the male individuals of bastard halibut (Paralichthys olivaceus) deficient in leptin receptor genes.

### DESCRIPTION OF EMBODIMENTS

An embodiment of the present invention relates to a method for promoting sexual maturation of fish.

In the present description, “fish” is not particularly limited. In the present description, the fish can include saltwater fish, freshwater fish, brackish water fish, diadromous fish, and the like.

The fish include, for example, fishes of the families Paralichthys, Tetraodontidae: puffers, Ostraciidae: box fishes, Sparidae: sea breams and porgies, Salmonidae, Cyprinidae, Ictaluroidea, Siluroidea, Bagroidea, Serranidae: sea basses, Cichlidae, Oryziidae: medakas, Monacanthidae, Osmeridae, Scombridae, Pleuronectidae, Carangidae, Lateolabrax, Moronidae, Latidae, Rachycentridae, Cynoglossidae, Anguillidae, Congridae, and the like.

Fishes of the family Paralichthys can include, for example, Paralichthys olivaceus (TEMMINCK et SCHLEGEL), fishes belonging to the genus Pseudorhombus (ocellated flounder (Pseudorhombus dupliciocellatus), roughscale flounder (Pseudorhombus oligodon), Pseudorhombus ctenosquamis, largetooth flounder (Pseudorhombus arsius), fivespot flounder (Pseudorhombus pentophthalmus), cinnamon flounder (Pseudorhombus cinnamoneus), central spotted flounder (Pseudorhombus levisquamis), and the like), fishes belonging to the genus Tarphops (small flounder (Tarphops oligolepis) and the like), fishes belonging to the genus Asterorhombus (intermediate flounder (Asterorhombus intermedius), fishes belonging to the genus Lepidopsetta (dusky sole (Lepidopsetta mochigarei)), fishes belonging to the genus Taeniopsetta (Indo-Pacific ocellated flounder (Taeniopsetta ocellata)), and the like. A preferred fish of the family Paralichthys is Paralichthys olivaceus (TEMMINCK et SCHLEGEL).

Fishes of the family Tetraodontidae can include, for example: fishes belonging to the genus Takifugu such as torafugu (Takifugu rubripes), purple puffer (Takifugu porphyreus), and grass puffer (Takifugu niphobles); fishes belonging to the genus Lagocephalus such as half-smooth golden pufferfish (Lagocephalus wheeleri); and the like.

Fishes of the family Ostraciidae can include, for example, fishes belonging to the genus Ostracion such as bluespotted boxfish (Ostracion immaculatus).

Fishes of the family Sparidae can include, for example: fishes belonging to the genus Pagrus such as red seabream (Pagrus major) and squirefish (Pagrus auratus); fishes belonging to the genus Acanthopagrus such as black porgy (Acanthopagrus schlegeli) and yellowfin seabream (Acanthopagrus latus); fishes belonging to the genus Dentex such as yellowback seabream (Dentex tumifrons); fishes belonging to the genus Sparus such as gilthead seabream (Sparus aurata); and the like.

Fishes of the family Salmonidae can include, for example: fishes belonging to the genus Oncorhynchus such as rainbow trout (Oncorhynchus mykiss), Chinook salmon (Oncorhynchus tshawytscha), cherry salmon (Oncorhynchus masou masou), red-spotted masu salmon (Oncorhynchus masou ishikawae), black kokanee (Oncorhynchus kawamurae), pink salmon (Oncorhynchus gorbuscha), and chum salmon (Oncorhynchus keta); fishes belonging to the genus Salmo such as brown trout (Salmo trutta), sockeye salmon (Oncorhynchus nerka), coho salmon (Oncorhynchus kisutch), and Atlantic salmon (Salmo salar); fishes belonging to the genus Salvelinus such as Dolly Varden (Salvelinus malma), Arctic char (Salvelinus alpinus), whitespotted char (Salvelinus leucomaenis), brook trout (Salvelinus fontinalis), and lake trout (Salvelinus namaycush); fishes belonging to the genus Hucho such as Sakhalin taimen (Parahucho perryi); and the like.

Fishes of the family Cyprinidae can include, for example, Honmoroko (Gnathopogon caerulescens), silver carp (Hypophthalmichthys molitrix), Eurasian carp (Cyprinus carpio), grass carp (Ctenopharyngodon idellus), bighead carp (Hypophthalmichthys nobilis), crucian carp (Carassius carassius), catla (Cyprinus catla), black carp (Mylopharyngodon piceus), mud carp (Cirrhinus molitorella), mrigal carp (Cirrhinus cirrhosus), catla (Catla catla), rohu (Labeo rohita), Wuchang bream (Megalobrama amblycephala), and the like.

Fishes of the superfamily Ictaluroidea can include, for example, American catfish (Ictalurus punctatus), blue catfish (Ictalurus furcatus), and the like.

Fishes of the superfamily Siluroidea can include, for example, Amur catfish (Silurus asotus), Lake Biwa catfish (Silurus biwaensis), rock catfish (Silurus lithophilus), Wels catfish (Silurus glanis), whitespotted clarias (Clarias fuscus), walking catfish (Clarias batrachus), and the like.

Fishes of the superfamily Bagroidea can include, for example, yellow catfish (Pseudobagrus fulvidraco), Mekong giant catfish (Pangasianodon gigas), basa (Pangasius bocourti), striped catfish (Pangasianodon hypophthalmus), and the likes.

Fishes of the family Serranidae can include, for example: fishes belonging to the genus Epinephelus such as convict grouper (Epinephelus septemfasciatus), longtooth grouper (Epinephelus bruneus), Hong Kong grouper (Epinephelus akaara), Malabar grouper (Epinephelus malabaricus), white grouper (Epinephelus aeneus), banded grouper (Epinephelus amblycephalus), areolate grouper (Epinephelus areolatus), duskytail grouper (Epinephelus bleekeri), pale margin grouper (Epinephelus bontoides), brown spotted grouper (Epinephelus chlorostigma), orange-spotted grouper (Epinephelus coiodes), blacktip grouper (Epinephelus fasciatus), brown-marbled grouper (Epinephelusfuscoguttatus), starry grouper (Epinephelus labriformis), giant grouper (Epinephelus lanceolatus), highfin grouper (Epinephelus maculatus), Malabar grouper (Epinephelus malabaricus), dusky grouper (Epinephelus marginatus), white-streaked grouper (Epinephelus ongus), camouflage grouper (Epinephelus polyphekadion), longfin grouper (Epinephelus quoyanus), sixbar grouper (Epinephelus sexfasciatus), Nassau grouper (Epinephelus striatus), greasy grouper (Epinephelus tauvina), and potato grouper (Epinephelus tukula); fishes belonging to the genus Cromileptes such as humpback grouper (Cromileptes altivelis); fishes belonging to the genus Plectropomus such as leopard coralgrouper (Plectropomus leopardus); and crossbreds among fishes of the family Serranidae.

Fishes of the family Cichlidae can include, for example, fishes of the genus Oreochromis such as Nile tilapia (Oreochromis niloticus), Mozambique tilapia (Oreochromis mossambicus), and Blue tilapia (Oreochromis aureus).

Fishes of the family Adrianichthyidae can include, for example, fishes of the genus Oryzias such as medaka (Oryzias latipes, Oryzias sakaizumii) and Javanese ricefish (Oryzias javanicus).

Fishes of the family Monacanthidae can include, for example, fishes of the genus Stephanolepis such as thread-sail filefish (Stephanolepis cirrhifer) and fishes of the genus Thamnaconus such as black scraper (Thamnaconus modestus).

Fishes of the family Osmeridae can include, for example: fishes of the subfamily Plecoglossinae such as ayu (Plecoglossus altivelis); fishes of the subfamily Hypomesinae such as Japanese smelt (Hypomesus nipponensis) and Japanese surfsmelt (Hypomesus japonicus); fishes of the subfamily Osmerinae such as Arctic rainbow smelt (Osmerus mordax dentex), Shishamo smelt (Spirinchus lanceolatus), and Japanese icefish (Salangichthys microdon); and the like.

Fishes of the family Scombridae can include, for example: fishes belonging to the genus Scombrini such as chub mackerel (Scomber japonicus), Atlantic mackerel (Scomber scombrus), and blue mackerel (Scomber australasicus); fishes belonging to the genus Thunnini such as Pacific bluefin tuna (Thunnus orientalis), Atlantic bluefin tuna (Thunnus thynnus), southern bluefin tuna (Thunnus maccoyii), bigeye tuna (Thunnus obesus), yellowfin tuna (Thunnus albacares), albacore (Thunnus alalunga), and longtail tuna (Thunnus tonggol); fishes belonging to the genus Euthynnus such as Kawakawa (Euthynnus affinis) and Little tunny (Euthynnus alletteratus); fishes belonging to the genus Katsuwonus such as skipjack tuna (Katsuwonus pelamis); fishes belonging to the genus Scomberomorini; fishes belonging to the genus Auxis; fishes belonging to the genus Sardini; fishes belonging to the genus Gymnosarda; and the like.

Fishes of the family Pleuronectidae can include, for example, littlemouth flounder (Pseudopleuronectes herzensteini), marbled flounder (Pleuronectes yokohamae), stone flounder (Kareius bicoloratus), Pacific halibut (Hippoglossus stenolepis), barfin flounder (Verasper moseri), and the like.

Fishes of the family Carangidae can include, for example: fishes belonging to the genus Seriola such as greater amberjack (Seriola dumerili), yellowtail amberjack (Seriola lalandi), almaco jack (Seriola rivoliana), and Japanese amberjack (Seriola quinqueradiata); fishes belonging to the genus Pseudocaranx such as Japanese jack mackerel (Trachurus japonicus) and hard-tail jack (Pseudocaranx dentex); fishes belonging to the genus Trachinotus such as snubnose pompano (Trachinotus blochii).

Fishes of the family Lateolabrax can include, for example, blackfin seabass (Lateolabrax latus), spotted seabass (Lateolabrax maculatus), and the like. Fishes of the family Moronidae includes, for example, European seabass (Dicentrarchus labrax) and the like.

Fishes of the family Latidae can include, for example, fishes of the genus Lates such as barramundi perch (Lates calcarifer) and Nile perch (Lates niloticus).

Fishes of the family Rachycentridae can include, for example, cobia (Rachycentron canadum) and the like.

Fishes of the family Cynoglossidae can include, for example, red tonguesole (Cynoglossus joyneri), tongue sole (Cynoglossus semilaevis), and the like.

Fishes of the family Anguillidae can include, for example, Japanese eel (Anguilla japonica), European eel (Anguilla anguilla), and the like.

Fishes of the family Congridae can include, for example, whitespotted conger (Conger myriaster), Beach conger (Conger japonicus), and the like.

In the present description, the fish may be true breds or crossbreds. The crossbreds can include, for example, hybrids derived from intergeneric crossing.

The “fish” preferably belongs to any of the families Paralichthys, Tetraodontidae, Sparidae, Serranidae, Monacanthidae, Cichlidae, Salmonidae, and Osmeridae. More preferably, the “fish” is preferably bastard halibut (Paralichthys olivaceus), torafugu (Takifugu rubripes), thread-sail filefish (Stephanolepis cirrhifer), ayu (Plecoglossus altivelis), chub mackerel (Scomber japonicus), rainbow trout (Oncorhynchus mykiss), Nile tilapia (Oreochromis niloticus) or Hong Kong grouper (Epinephelus akaara).

The “fish” is preferably cultured fish. In addition, the “farmed fish” can include, for example, fish that are bred for purposes of food, reproduction, decoration, and the like.

In the present description, “sexual maturity” means that ovulation is observed for female individuals and spermiation is observed for male individuals.

“Promoting sexual maturity” means appearance of an ability of spawning (or ovulation) or spermiation that is earlier than the shortest age at maturity of the average mature individual of the same species in nature or under normal aquaculture conditions.

In the case where the shortest age at maturity of the individual of the same species is x months, for example, to be “earlier than the shortest age at maturity” means [x months−one month] or shorter (“−” means subtraction). Preferably, to be “earlier than the shortest age at maturity” means [x months×0.2] months to [x months×0.8] months, further preferably [x months×0.45] months to [x months×0.75] months, even further preferably [x months×0.45] months to [x months×0.6] months.

More specifically, according to Non Patent Literature 1, female individuals of bastard halibut (Paralichthys olivaceus) are capable of ovulation at 24 months of age or older, except for a very few individuals. In addition, in Non Patent Literature 1, one individual having ovaries at the full maturity stage with hydrated eggs (ovarian eggs) at less than 24 months of age and one individual having ovaries at the tertiary yolk stage have been identified, but both of the individuals are described as being immediately before 2 years of age. Furthermore, it is not reported that fertilized eggs that can be produced are obtained. Thus, it cannot be said that it does not indicate the shortest age at maturity of the individual. Based on the above, it is considered that these individuals are older than the age of 24 months when actually spawning. Thus, in the present description, in the case of bastard halibut (Paralichthys olivaceus), “promoting sexual maturity” specifically means, in the case of the female individuals, that the first ovulation is possible within 24 months, preferably 10 to 18 months, more preferably 11 to 16 months, and even more preferably 11 to 12 months, with a date of fertilization as day 0 and a month of fertilization as month 0. In addition, bastard halibut (Paralichthys olivaceus) are asynchronous multiple spawners. Thus, the first ovulation means the first day of ovulation at the time when the individual enters a first spawning period thereof. For the male individuals, on the other hand, “promoting sexual maturity” means that the first spermiation is possible within 12 months, preferably from 5 to 12 months, and more preferably from 8 to 12 months. Here, “one month” at the time of counting the age in month is defined that day 0 is a starting date and day 30 counted from day 0 is a last date.

Here, the term “ovulation” means discharge of hydrated eggs from an ovary into an ovarian cavity and means a state where the eggs can be discharged with an abdominal pressure. The term “spawning” means discharge of the eggs by a female individual involved in mating behavior.

The age in months of fish can be counted from the month of fertilization as described above. Even when the month of fertilization is not clear, the age can be assessed on the basis of the number of opaque bands on a surface of an otolith, assuming January 1 as an age reference date. That is, by this standard, the age in the state of the promoted sexual maturity is less than two years of age in a case of bastard halibut (Paralichthys olivaceus), for example.

In the present description, the method for promoting sexual maturation of fish includes a step of suppressing functional expression of at least one of a leptin receptor and leptin.

Here, mRNA of the leptin receptor or leptin mRNA for each fish is not limited as long as the mRNA is transcribed from the leptin receptor or a leptin gene present in a genome of each of the fish. That is, the mRNA of the leptin receptor or the leptin for each fish can contain a variant or the like that is transcribed from the leptin receptor or the leptin gene.

For example, a leptin receptor gene of bastard halibut (Paralichthys olivaceus) is registered as Gene ID (NCBI): 109636534. The leptin genes of bastard halibut (Paralichthys olivaceus) are registered as Gene IDs (NCBI): 109624291 and 109632763.

An mRNA sequence of the leptin receptor of bastard halibut (Paralichthys olivaceus) is registered as NCBI Reference Sequence: XM_020098286.1. In addition, mRNA sequences of the leptin of bastard halibut (Paralichthys olivaceus) are registered as Reference Sequences: NW_017859644 and NW_017859660. As specific examples of various fishes, the leptin receptor genes are shown in Table 1 below, and the leptin genes are shown in Table 2 below.

TABLE 1

| | | GenBank ID | Gene ID |
|---|---|---|---|
| Species Name | Scientific Name | (NCBI) | (NCBI) |
| Tiger pufferfish | Takifugu rubripes | XM_029827573 | 100174783 |
| Gilthead seabream | Sparus aurata | XM_030433806 | 115591642 |
| Rainbow trout | Oncorhynchus mykiss | XM_021604608 | 110524724 |
| Chinook salmon | Oncorhynchus tshawytscha | XM_042321277 | 112250617 |
| Brown trout | Salmo trutta | XM_029707951 | 115158716 |
| Arctic char | Salvelinus alpinus | XM_024004689 | 111975995 |
| Atlantic salmon | Salmo salar | NM_001164765 | 100303650 |
| Common carp | Cyprinus carpio | XM_019063286 | 109045413 |
| Grass carp | Ctenopharyngodon idella | JQ080547 | |
| Silver carp | Hypophthalmichthys molitrix | KM068181 | |
| Channel catfish | Ictalurus punctatus | XM_017480265 | 108272094 |
| Yellow catfish | Pelteobagrus fulvidraco | XM_027147262 | 113643163 |
| Yellow catfish | Tachysurus fulvidraco | XM_047814074 | 113643163 |
| Striped catfish | Pangasianodon hypophthalmus | XM_034310139 | 113537103 |
| Channel catfish | Ictalurus punctatus | XM_047158461 | 108272094 |
| Giant grouper | Epinephelus lanceolatus | XM_033622223 | 117254167 |
| Orange-spotted grouper | Epinephelus coioides | JX406149 | |
| Nile tilapia | Oreochromis niloticus | XM_005460448 | 100702842 |
| Blue tilapia | Oreochromis aureus | XM_005460448 | 100702842 |
| Mozambique tilapia | Oreochromis mossambicus | KC354703 | |
| Tongue sole | Cynoglossus semilaevis | XM_025065905 | 103395564 |
| Chub mackerel | Scomber japonicus | KP635451 | |
| Greater amberjack | Seriola dumerili | | 111222157 |
| Yellowtail amberjack | Seriola lalandi | | 111645375 |
| Japanese seabass | Lateolabrax japonicus | KF850512 | |
| European seabass | Dicentrarchus labrax | KF918755 | |
| Barramundi | Lates calcarifer | XM_018696399 | 108897025 |
| European eel | Anguilla anguilla | LN558791 | |

TABLE 2

| | | GenBank ID | Gene ID |
|---|---|---|---|
| Species Name | Scientific Name | (NCBI) | (NCBI) |
| Tiger pufferfish | Takifugu rubripes | NM_001032725 | 548631 |
| Nile tilapia | Oreochromis niloticus | NM_001301050 | 100704227, |
| | | NM_001301049 | 100704338 |
| Rainbow trout | Oncorhynchus mykiss | XM_021564028 | 110490587 |
| Chinook salmon | Oncorhynchus tshawytscha | XM_024379558 | 112218616 |
| Brown trout | Salmo trutta | XM_029696522 | 115152051 |
| Arctic char | Salvelinus alpinus | XM_023972149 | 111953061 |
| Atlantic salmon | Salmo salar | XM_014124944 | 106561227 |
| Channel catfish | Ictalurus punctatus | XM_017465190 | 100304688 |
| Yellow catfish | Tachysurus fulvidraco | XM_027161401 | 11365239 |
| Chub mackerel | Scomber japonicus | KP635450 | |
| | | KP635449 | |
| Greater amberjack | Seriola dumerili | XM_022749646 | 111224940 |
| Yellowtail amberjack | Seriola lalandi | XM_023408217 | 111656484 |

The method for suppressing the functional expression of at least one of the leptin receptor and the leptin is not limited. For example, the functional expression is suppressed by introducing a loss-of-function mutation in at least one of the leptin receptor and the leptin, by suppressing expression of at least one gene of the leptin receptor and the leptin, or by neutralizing a function of a protein of at least one of the leptin receptor and the leptin.

A method for introducing the loss-of-function mutation in at least one of the leptin receptor and the leptin is not limited as long as the loss-of-function mutation can be introduced into a target gene in the genome of the target fish. Such a method is known. Examples of the method for introducing the loss-of-function mutation can include site-directed mutagenesis methods, such as a genome editing method and a homologous recombination method, and a random mutagenesis method.

The genome editing method can include a method for introducing a protein and a nucleic acid, which constitute a genome editing technique, or vectors encoding these protein and nucleic acid. Examples of the protein can include clustered regularly interspaced short palindromic repeats (CRISPR) enzymes. More specifically, examples of the CRISPR enzymes can include Cas1, Cas1B, Cas2, Cas3, Cas4, Cas5, Cas6, Cas7, Cas8, Cas9, Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. Examples of the nucleic acids can include crRNA, tracrRNA, and a single-stranded nucleic acid in which crRNA and tracrRNA are linked via a linker. In this case, for example, the nucleic acid is designed, for example, such that a nucleotide sequence to anneal to a target sequence in the crRNA is complementary to a nucleotide sequence encoding the leptin receptor and the leptin gene. A single type of the nucleic acid may be used alone, or two or more types of the nucleic acids may be used in combination. The genome editing systems using the CRISPR enzyme can include a clustered regularly interspaced short palindromic repeats/CRISPR associated protein 9 (CRISPR/Cas9) system. As another genome editing system, at least one method can be selected from a CompoZr zinc-finger nuclease (ZFN) system and a TAL effector nuclease (TALEN) system. Preferably, the genome editing system is the CRISPR/Cas9 system that introduces Cas9 as mRNA or a protein and introduces gRNA as sgRNA or crRNA and tracrRNA. In a vector-based CRISPR/Cas9 system, the nucleic acid encoding CRISPR and the nucleic acid encoding Cas9 may be on different vectors or on one vector. A promoter for the CRISPR function is not particularly limited. However, a U6 promoter is preferred. A promoter for the Cas9 function is not particularly limited. However, a promoter expressed in a mammalian cell, such as a Cytomegalovirus promoter, is preferred. Preferably, a commercially available vector such as a pX330-U6-Chimeric_BB-CBh-hSpCas9 vector can be used as the CRISPR/Cas9 system.

A sequence that targets the leptin receptor gene or the leptin gene incorporated into a CRISPR sequence (hereinafter also referred to as a “target sequence”) is not particularly limited as long as such a sequence can be transcribed by the CRISPR/Cas9 system into guide RNA (also referred to as gRNA, sgRNA, or crRNA) or introduced, as the guide RNA that contains the complementary sequence to the target sequence, into cells to recombine the leptin receptor gene or the leptin gene. In general, it is said that, as the target sequence, a sequence of about 20 bases in a 5′ upstream region of a nucleotide sequence “NGG” (a PAM sequence: N is any of nucleotide A, G, T, C), which is present in the leptin receptor gene or the leptin gene, can be selected. The target sequence can be designed by using any of known design tools published by an Optimized CRISPR design tool (a website of Zhang Lab at Massachusetts Institute of Technology (http://crispr.mit.edu/)), E-CRISP (http://www.e-crisp.org/E-CRISP/(German Cancer Research Center)), ZiFiT Targeter (http://zifit.partners.org/ZiFit/(Zing Finder Consortium)), Cas9 design (http://cas9.cbi.pku.edu.cn (Peking University)), CRISPRdirect (http://crispr.dbcls.jp (the University of Tokyo)), CRISPR-P (http://cbi.hzau.edu.cn/crispr/(Huazhong Agricultural University)), CRISPR RGEN Tools (http://www.rgenome.net/(Seoul National University)), and the like.

An example of the sequence that targets the leptin receptor gene can include 5′-CAGTGCGTCCTTCGGAGACTCGG-3′ (SEQ ID NO: 1).

In addition, preferably, when single nucleotide polymorphisms (SNPs) exist in the PAM sequence, such a sequence is preferably avoided. In regard to the target sequence, when the SNPs of an individual are known, the sequence is preferably optimized for each of the SNPs.

In the 5′-terminal region of the target sequence, the sequence may be one, two, three, or four bases shorter, preferably, one, two, or three bases shorter.

In the CRISPR/Cas9 system, a gene, as a vector, may be introduced into cells, or a combination of a gRNA, a crRNA, a trans-activating crRNA (tracrRNA), and an RNA encoding Cas9 synthesized by artificial synthesis or in vitro transcription may be introduced into the cells. Alternatively, a combination of a Cas9 protein and a guide RNA may be introduced into the cell.

Furthermore, in the genome editing system, a donor oligo DNA such as a single-stranded oligos (ssODNs) may be co-introduced into the cell. The ssODNs can be designed according to a known method.

The genome editing system can perform microinjection into cytoplasm of a fertilized egg, preferably, a fertilized egg at a one-cell stage. For example, when being introduced, the Cas9 protein can be injected in a range of 5 pg to 100 pg, preferably 10 pg to 80 pg, more preferably 10 pg to 50 pg per fertilized egg. At this time, the guide RNA can be injected in a range of 0.1 pg to 50 pg, preferably 0.5 pg to 20 pg, more preferably 1 pg to 5 pg.

Example 1 described below can be referred for a method for introducing a mutation using the genome editing technique, for example.

Examples of the random mutagenesis method can include: treatment with radiation such as α-rays, β-rays, γ-rays, and X-rays; a chemical substance treatment using a mutagenic agent such as ethyl methanesulfonate (EMS) or ethynylnitrosourea (ENU); and a heavy ion beam treatment.

The method for suppressing the expression of a gene of at least one of the leptin receptor and the leptin can include a method for introducing at least one type selected from a group including RNA molecules targeting at least one mRNA selected from the leptin receptor and the leptin, or vectors capable of expressing such RNA molecules. The term “RNA molecule targeting at least one mRNA selected from the leptin receptor and the leptin” is not limited as long as the RNA molecule targets the leptin receptor or leptin mRNA and can suppress the expression of the leptin receptor or leptin protein. Examples of the RNA molecules can include those having a degrading action on the target mRNA such as a siRNA, a shRNA, a dsRNA, a miRNA and/or those suppressing translation of the target mRNA. A person skilled in the art can design sequences of these RNA molecules appropriately using a known method on the basis of information on a nucleotide sequence of the target gene. The RNA molecules may be prepared on the basis of a known method, or the RNA molecules available in the market may be obtained and used. As the RNA molecules, the siRNA, the shRNA, and the miRNA are preferred, and the siRNA and the shRNA are especially preferred. The vectors capable of expressing the RNA molecules targeting the leptin receptor or leptin mRNA are not particularly limited as long as the vectors can express the RNA molecules suppressing the expression of the leptin receptor or leptin protein in an individual body or in a cell. An example of such a vector can be a hairpin RNA expression vector. The hairpin RNA expression vectors at least include: a sense-strand DNA nucleotide sequence having the same sequence as a sense strand of the target mRNA (however, uracil of the mRNA is replaced to thymine) on a downstream of a promoter sequence suitable for expression of short-strand RNA such as a U6 promoter; a loop nucleotide sequence that forms a loop structure after transcription; an antisense-strand DNA nucleotide sequence that can complementarily bind in whole or in part to the sense-strand DNA nucleotide sequence; and a terminator sequence, for example. Examples of the vectors can include a plasmid vector, an adenovirus vector, a retrovirus vector, and a lentivirus vector.

At least one RNA molecule selected from the group including the siRNA, the shRNA, and the miRNA can be injected by microinjection in a range of 5 pg to 100 pg, preferably 10 pg to 80 pg, more preferably 10 pg to 50 pg per fertilized egg. At least one RNA molecule selected from the group including the siRNA, the shRNA, and the miRNA, or the vector capable of expressing such an RNA molecule can be injected by microinjection in a range of 5 pg to 100 pg, preferably 10 pg to 80 pg, more preferably 10 pg to 50 pg per fertilized egg. The vector can be linearized when necessary.

The function of the protein can be suppressed using at least one type selected from a group including antibodies that specifically bind to one of the leptin receptor and the leptin to suppress the functional expression of the binding target by such binding. Such an antibody may be a polyclonal antibody or a monoclonal antibody. A person skilled in the art can appropriately prepare both the polyclonal antibody and the monoclonal antibody using a known method. In addition, the antibody may be an antibody fragment such as Fab, F(ab)2, a diabody, scFv, a minibody, a peptibody, or a mimetibody.

The antibody can be injected in a range of 5 pg to 100 pg, preferably 10 pg to 80 pg, more preferably 10 pg to 50 pg per fertilized egg.

Hatchlings hatched from fertilized eggs and fries are reared for one to five months, and an individual in which a mutation is induced in the leptin receptor gene is selected by a mutation analysis method, such as a heteroduplex mobility analysis, Cel-1 assay, or a T7 endonuclease assay sequence analysis. The individual is reared at a water temperature of about 15.0 to 22.0° C. from 5 to 16 months of age. The daylight hours can be set under the natural condition. Other rearing conditions can be set under general aquaculture conditions. The same food as that generally fed under the general aquaculture conditions can be used.

For example, in the case of bastard halibut (Paralichthys olivaceus), hatchlings hatched from fertilized eggs and fries are reared at the water temperature of about 14.4 to 16.4° C. until 2.6 months of age. Individuals with the induced mutation in the leptin receptor gene are selected by the heteroduplex mobility analysis. The individuals are reared at the water temperature of about 16 to 19.3° C. until 5.8 months of age, and then reared at the water temperature of about 14.3 to 20.6° C. until 14.7 months of age. The individuals are reared at the water temperature of about 15.6 to 22.2° C. from 14.7 months of age. The daylight hours can be set under the natural condition. Other rearing conditions can be set under general aquaculture conditions. The same food as that generally fed under the general aquaculture conditions can be used.

### EXAMPLES

An aspect of the present invention will be described in detail in Examples below. However, the present invention should not be construed as being limited to Examples.

1. Preparation of Bastard Halibut (Paralichthys olivaceus) Deficient in Leptin Receptor Gene

Bastard halibut (Paralichthys olivaceus) deficient in a portion of the leptin receptor gene were prepared and compared with wild bastard halibut (Paralichthys olivaceus) to examine promotion of sexual maturity.

### (1) Preparation of Fertilized Eggs

Eggs (unfertilized eggs) and sperms were respectively collected from sexually mature male and female bastard halibut (Paralichthys olivaceus) by applying a pressure to the abdomens. The obtained eggs and sperms were artificially inseminated to obtain the fertilized eggs.

### (2) Introduction of Mutation

A mutation was introduced using the Cas9 protein and the guide RNA. A sequence of the target gene is 5′-CAGTGCGTCCTTCGGAGACTCGG-3′ (SEQ ID NO: 1), and the RNA was synthesized by Fasmac Co., Ltd.

The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage obtained in (1) above. The fertilized eggs transfected with the Cas9 protein and the guide RNA were reared and hatched under normal rearing conditions to obtain an F0 generation.

### (3) Preparation of F1 Generation

The F0 individuals obtained in (2) above were sexually matured to obtain individuals in F1 offspring generation homozygously deficient in the leptin receptor genes and heterozygously deficient in the leptin receptor genes. An altered sequence of the target gene caused deficiency of 8 bases (5′-TCGGAGAC-3′) in (1) above, resulting in loss of the function of the leptin receptor gene.

2. Preparation of Bastard Halibut (Paralichthys olivaceus) Deficient in Leptin Receptor Gene and and Observation of Sexual Maturation Period of Such Bastard Halibut (Paralichthys olivaceus)

### (1) Rearing Conditions

The hatchlings of the F0 generation described above (2) were reared in a 0.5-ton tank at the water temperature of 14.4 to 16.4° C. until 2.6 months of age. The individuals were identified at 2.6 months of age, and each of the individuals was labeled with an individual identification tag. Then, 34 individuals were selected for mutation induced in the leptin receptor genes by the heteroduplex mobility analysis. The fish were reared in a 1-ton tank at the water temperature of 16 to 19.3° C. until 5.8 months of age, and then reared in a 3-ton tank at the water temperature of 14.3 to 20.6° C. until 14.7 months of age. From 14.7 months of age, the fish were reared in a 10-ton tank at the water temperature of 15.6 to 22.2° C. The daylight hours were set under the natural condition.

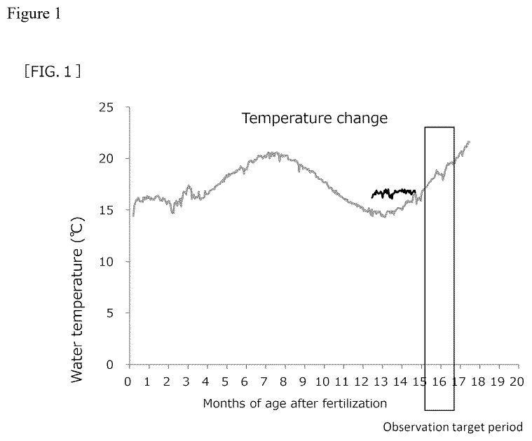

FIG. 1 shows the temperature condition during rearing.

### (2) Results

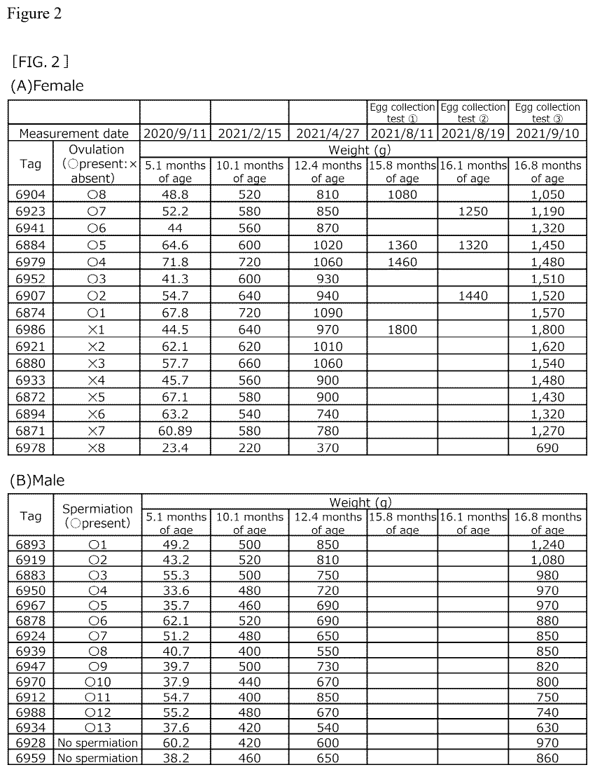

FIG. 2A shows presence or absence of ovulation in female individuals of bastard halibut (Paralichthys olivaceus) deficient in the leptin receptor genes in the F0 generation and weight changes at 5.1, 10.1, 12.4, 15.8, 16.1, and 16.8 months of age. Among a half of 16 individuals, the ovulation was observed from 15.8 to 16.1 months of age. It was confirmed that the eggs were fertilized, developed, and hatched normally.

FIG. 2B shows presence or absence of spermiation in the male individuals of bastard halibut (Paralichthys olivaceus) deficient in the leptin receptor genes in the F0 generation and the weight changes at 5.1, 10.1, 12.4, 15.8, 16.1, and 16.8 months of age. Among 13 of 15 individuals, the spermiation was observed at 10.5 months of age. It was confirmed that the sperms moved normally.

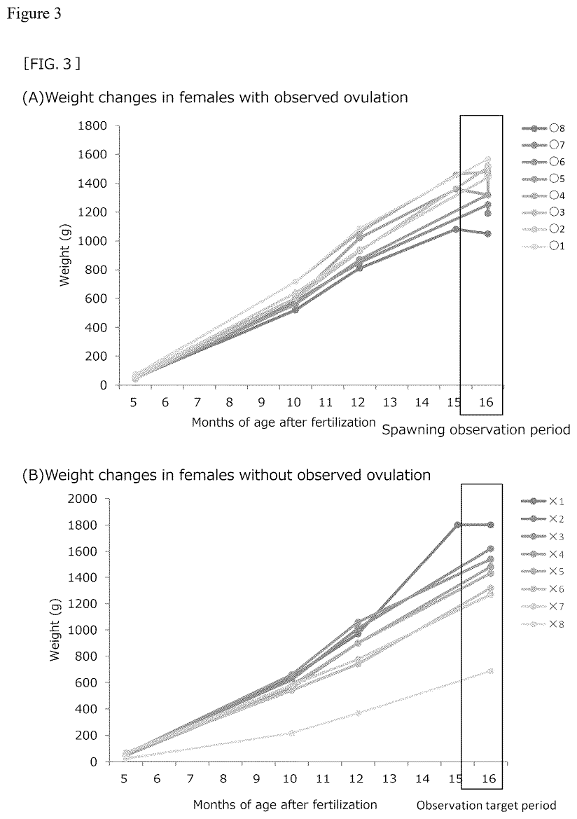

FIGS. 3A and 3B each show the weight changes in the female individuals of bastard halibut (Paralichthys olivaceus) deficient in leptin receptor genes in the F0 generation. FIG. 3A shows a group in which the ovulation has been observed, and FIG. 3B shows a group in which the ovulation has not been observed.

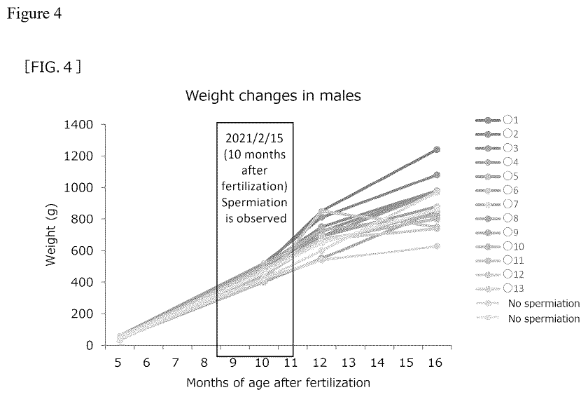

FIG. 4 shows the weight changes in the male individuals of bastard halibut (Paralichthys olivaceus) deficient in leptin receptor genes in the F0 generation.

It is reported that it generally takes two years for the individual female bastard halibut (Paralichthys olivaceus) to become sexually mature and ready to ovulate. It is also reported that it generally takes one year for the individual male bastard halibut (Paralichthys olivaceus) to become sexually mature and ready for spermiation.

The deficiency of the leptin receptor gene resulted in a shorter time to the sexual maturity in both of the female and male individuals compared to the generally reported time to the sexual maturity.

This indicated that inhibition of signal transduction by binding of the leptin receptor and the leptin as a ligand thereof in the body of bastard halibut (Paralichthys olivaceus) implemented an effect of promoting the sexual maturation of bastard halibut (Paralichthys olivaceus).

### 3. Sexual Maturation of Male Individuals in F1 Generation Homozygously Deficient in Leptin Receptor

The sexual maturation was confirmed among the male individuals in the F1 generation homozygously deficient in the leptin receptors (the individuals as the offspring of the fish in Example). Of the six male individuals, the spermiation was observed in the six individuals. It was also confirmed that the sperms moved normally. The male individuals were five months of age, and the time required for the sexual maturation was significantly reduced in comparison with bastard halibut (Paralichthys olivaceus) which were produced in Section 1 above and became ready for spermiation at 10 months of age.

### 4. Sexual Maturation of Female Individuals in F1 Generation Heterozygously Deficient in Leptin Receptor

The sexual maturation was confirmed among the individuals in the F1 generation heterozygously deficient in the leptin receptors (the individuals as the offspring of the fish in Example). Of the 26 female individuals, spawning was observed in 1 individual. The female individual was 358 days of age, and the time required for the sexual maturation was significantly reduced in comparison with bastard halibut (Paralichthys olivaceus) which were produced in Section 1 above and became ready for ovulation at 1.4 years of age.

5. Preparation of Torafugu (Takifugu rubripes) Deficient in Leptin Receptor Gene and Observation of Sexual Maturation Period of Such Torafugu (Takifugu rubripes)

Torafugu (Takifugu rubripes) deficient in the leptin receptor genes were produced and compared with wild torafugu (Takifugu rubripes) to examine the promotion of the sexual maturity.

### (1) Preparation of Fertilized Eggs

Eggs (unfertilized eggs) and sperms were respectively collected from sexually mature male and female torafugu (Takifugu rubripes) by applying the pressure to the abdomens. The obtained eggs and sperms were artificially inseminated to obtain the fertilized eggs.

### (2) Introduction of Mutation

A mutation was introduced using the Cas9 mRNA and the guide RNA. A sequence of the target gene is 5′-GTAGATGGAACAGCACACAGTGG-3′ (SEQ ID NO: 2). The mutation was introduced into the leptin receptor gene by microinjection of a solution, which was prepared to have 2 to 10 pg of the Cas9 mRNA and 1 to 5 pg of the guide RNA, into cytoplasm of the fertilized egg at the one-cell stage. The fertilized eggs transfected with the Cas9 mRNA and the guide RNA were reared and hatched under normal rearing conditions to obtain the F0 generation.

### (3) Production of Phylesis

The individuals obtained in (2) above were sexually matured to obtain individuals in the offspring generation homozygously deficient in the leptin receptor genes. An altered sequence of the target gene caused deficiency of four bases in (1) above, resulting in loss of the function of the leptin receptor gene. The deficient sequence of the target gene is 5′-GTAGATGGAACAG----CAGTGG-3′ (SEQ ID NO: 3).

### (4) Observation of Sexual Maturation Period

The individuals in the F2 generation homozygously deficient in the leptin receptor genes in (4) above were reared at the natural water temperature and under natural sunlight conditions until 2 years of age. Those individuals were reared at the water temperature of 17° C. and with 14 daylight hours and 10 hours of darkness only in a maturation period. The female individuals were observed to spawn at two years of age.

6. Preparation of Thread-Sail Filefish (Stephanolepis cirrhifer) Deficient in Leptin Receptor Gene and Observation of Sexual Maturation Period

Thread-sail filefish (Stephanolepis cirrhifer) deficient in the leptin receptor genes (this gene sequence is unknown, and thus a nucleotide sequence will be described below) were produced to examine the promotion of the sexual maturity in comparison with wild thread-sail filefish (Stephanolepis cirrhifer).

### (1) Preparation of Fertilized Eggs

Eggs (unfertilized eggs) and sperms were respectively collected from sexually mature male and female thread-sail filefish (Stephanolepis cirrhifer) by applying the pressure to the abdomens. The obtained eggs and sperms were artificially inseminated to obtain fertilized eggs.

### (2) Introduction of Mutation

A mutation was introduced using the Cas9 protein and the guide RNA. A sequence of the target gene is 5′-CCCTCACGCAGTTCCTGAACGCG-3′ (SEQ ID NO: 4). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

7. Preparation of Ayu (Plecoglossus altivelis) Deficient in Leptin Receptor Gene and Observation of Sexual Maturation Period

Ayu (Plecoglossus altivelis) deficient in the leptin receptor genes (this gene sequence is unknown, and thus a nucleotide sequence will be described below) were produced to examine the promotion of the sexual maturity in comparison with wild ayu (Plecoglossus altivelis).

### (1) Preparation of Fertilized Eggs

Eggs (unfertilized eggs) and sperms were respectively collected from sexually mature male and female ayu (Plecoglossus altivelis) by applying the pressure to the abdomens. The obtained eggs and sperms were artificially inseminated to obtain fertilized eggs.

### (2) Introduction of Mutation

A mutation was introduced using the Cas9 protein and the guide RNA. A sequence of the target gene is 5′-AAAGTGTCCAATCAGGAAGGAGG-3′ (SEQ ID NO: 5). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

8. Preparation of Chub Mackerel (Scomber japonicus) Deficient in Leptin Receptor Gene and Observation of Sexual Maturation Period

Chub mackerel (Scomber japonicus) deficient in the leptin receptor genes (this gene sequence is unknown, and thus a nucleotide sequence will be described below) were produced to examine the promotion of the sexual maturity in comparison with wild chub mackerel (Scomber japonicus).

### (1) Preparation of Fertilized Eggs

Eggs (unfertilized eggs) and sperms were respectively collected from sexually mature male and female chub mackerel (Scomber japonicus) by applying the pressure to the abdomens. The obtained eggs and sperms were artificially inseminated to obtain fertilized eggs.

### (2) Introduction of Mutation

A mutation was introduced using the Cas9 protein and the guide RNA. A sequence of the target gene is 5′-TTCTGGTCTGCTGAGATATGAGG-3′ (SEQ ID NO: 6). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

9. Preparation of Rainbow Trout (Oncorhynchus mykiss) Deficient in Leptin Receptor Gene and Observation of Sexual Maturation Period

Rainbow trout (Oncorhynchus mykiss) deficient in the leptin receptor genes were produced and compared with wild rainbow trout (Oncorhynchus mykiss) to examine the promotion of the sexual maturity.

### (1) Preparation of Fertilized Eggs

Eggs (unfertilized eggs) and sperms were respectively collected from sexually mature male and female rainbow trout (Oncorhynchus mykiss) by applying the pressure to the abdomens. The obtained eggs and sperms were artificially inseminated to obtain fertilized eggs.

### (2) Introduction of Mutation

The mutation was introduced using the Cas9 protein and one of guide RNA #1 and guide RNA #2. Sequences of the target gene are 5′-CCCTGCTCTGCCTCACCTGTGGA-3′ (SEQ ID NO: 7) and 5′-AGGGAGAGTTGATTCTGAGCTGG-3′ (SEQ ID NO: 8). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

10. Preparation of Nile tilapia (Oreochromis niloticus) Deficient in Leptin Receptor Gene and Observation of Sexual Maturation Period

Tilapia deficient in the leptin receptor genes were produced and compared with wild tilapia to examine the promotion of the sexual maturity.

### (1) Preparation of Fertilized Eggs

Eggs (unfertilized eggs) and sperms were respectively collected from sexually mature male and female tilapia by applying the pressure to the abdomens. The obtained eggs and sperms were artificially inseminated to obtain fertilized eggs.

### (2) Introduction of Mutation

The mutation was introduced using the Cas9 protein and one of the guide RNA #1 and the guide RNA #2. Sequences of the target gene are 5′-CTCTCTGCGTTCAGGTGCCGTGG-3′ (SEQ ID NO: 9) and 5′-CCTTCAGCCCACCTTGGTGTGGA-3′ (SEQ ID NO: 10). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

11. Preparation of Hong Kong Grouper (Epinephelus akaara) Deficient in Leptin Receptor Gene and Observation of Sexual Maturation Period

Hong Kong grouper (Epinephelus akaara) deficient in the leptin receptor genes (this gene sequence is unknown, and thus a nucleotide sequence will be described below) were produced to examine the promotion of the sexual maturity in comparison with wild Hong Kong grouper (Epinephelus akaara).

### (1) Preparation of Fertilized Eggs

Eggs (unfertilized eggs) and sperms were respectively collected from sexually mature male and female Hong Kong grouper (Epinephelus akaara) by applying the pressure to the abdomens. The obtained eggs and sperms were artificially inseminated to obtain fertilized eggs.

### (2) Introduction of Mutation

The mutation was introduced using the Cas9 protein and the guide RNA. A sequence of the target gene is 5′-TCCACTCCTGAGTGCACTGCAGG-3′ (SEQ ID NO: 11). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

12. Preparation of Honmoroko (Gnathopogon caerulescens) Deficient in Leptin Receptor Gene and Observation of Sexual Maturation Period

Honmoroko (Gnathopogon caerulescens) deficient in the leptin receptor genes (this gene sequence is unknown, and thus a nucleotide sequence will be described below) were produced to examine the promotion of the sexual maturity in comparison with wild Honmoroko (Gnathopogon caerulescens).

### (1) Preparation of Fertilized Eggs

Eggs (unfertilized eggs) and sperms were respectively collected from sexually mature male and female Honmoroko (Gnathopogon caerulescens) by applying the pressure to the abdomens. The obtained eggs and sperms were artificially inseminated to obtain fertilized eggs

### (2) Introduction of Mutation

The mutation was introduced using the Cas9 protein and one of the guide RNA #1, the guide RNA #2, and the guide RNA #3. Sequences of the target genes are 5′-GACTGAATGGCTCTGGATACTGG-3′ (SEQ ID NO: 12), 5′-AAGATTAAGCACCAGGCCTCAGG-3′ (SEQ ID NO: 13), and 5′-ACTCGGCTTCACCTGAGGCCTGG-3′ (SEQ ID NO: 14). The mutation was introduced into the leptin receptor gene by microinjection of a solution, which was prepared to have 10 to 50 pg of the Cas9 protein and 1 to 5 pg of the guide RNA, into cytoplasm of the fertilized egg at the one-cell stage. The fertilized eggs transfected with the Cas9 protein and the guide RNA were reared and hatched under normal rearing conditions to obtain an F0 generation.

### (3) Screening for Mutagenesis and Observation of Promoted Sexual Maturation

For the individuals obtained in (2) above, a segment near the target sequence was amplified by PCR, and the promotion of the sexual maturation was observed among the individuals in which the mutation was introduced.

### <Leptin Receptor Gene Sequences>

Thread-sail filefish (Stephanolepis cirrhifer)

(SEQ ID NO: 15)
tacgcccgccgcatgcgtgtcaaaggtcaaagccgtcatgtgacg
aggctgttggtccctcacgcagTTCCTGAACGCGGCCCGGACTTC
TGGAGGATTCTCCAAGACCATCCGAACAGACCTCAGACCAAAGTC
ACCCTGTTGTTCAAGgaagtcgacacgtcaggtctctcgtactgt
gtggatggttttctaatccagcaccggagctccagtggctctgtg
acaaccgagcggatcgagccggccgcctccttcagcttcgactgg
aaccagaggcccgagaatgtgactgtgatggcctacaacagtttg
gggaactccagcaacaacagcttcatggtgctggacagacagccc
aaacgtcgctgcgtgcgCTCCTTCAGCGTGCAGGTGGTCAACCCA
TCCTGCGTCTCCCTGACGTGGACGCTGTTTGAGAACTGCTCCACC
CCCGACTTCATGGTGGTCCAGTGGTCCTCGCGGAGGCACCAGGGC
TCCGCCCACCACCGCAGGCTGCAGGGCGGACACACCTGGGNNNNN
NNNNNNNNNNNNNNNNNNNNNNNNNNNNNNNNNNNNNNNNNNNNN
NNNNNNNNNNNNNNNNNNNNNNNNNNNNNNNNNNNNNNNNNNNNN
NNNNNNNNNNNNNNNNNNNNNNNNNNNNNCGAAAGAGGAGAAGCT
GCCGTCTACCAGATGCTGCTGATCGTCTTCTTCCTCTCTGTCGTC
CTGTTTGTCACCCTCATCATCTCGCAGCAAAAaatgaagaagttt
gtgtggaaggaggtgcccgaccccagcaagtgctcctgggccaaa
ggagtggacTTCAGAAGGGTGGACCCTCTTGACCACATGTTCCGT
CCTGCCGACGATCTCCCAGCCAGGCCGCTGCTGCTGCTGCCGCTT
GAAAACATCTCCAAATTGGTCGTCATGGACAAGATAGACCTGTCA
GTGCTGAACGTAGACGTGGTCCAAGACCCCGCAGATCCAGTCGAC
TCGGAGGCCGACTGCCCTCAGCCACTTCCTGCTTCAGAGCCTCCA
TTCGTCCTCGACCTGGACGCTTTGACCAGCTTCAAACCAGAACAG
GAAGACCCGGCGAGGAAGGAGAACTCGGCCCAGTCTTTTGTCACC
TACTCCACTGTGCTGCTGCAGGATCCGCAGCAGCAGCACCAGCAG
CAGCACTACAAAGACGGCAGCAGCGGCAGTAGCAGCAGCAGCGAC
GAAGGAAACTTCTCGGCCAACAACTCGGACATCTCAGGGTCGTTC
CAGGGCGGCTTGTGGGAGCTGGACGACCCGCGGCGCTCCTGCTCC
TTCAACTCCGTGGAGCAGCTGTCGGAGGCGTCGGAGCAGGAGGAC
GAGAGCCGAGTCAGAGGCGAGAAGGACTTGTTCTACCTGGGGGTG
GAGGGGTACCCTGGGGACGAGAGCGAGGACGAGGAGGAGAAGGAT
GAGACGGACACCGAGCTGCTGAAGAGTGTCCCGGGCAGGGAGGAA
CGTCCCGCCGAGTCGCACCCTCTGCTGGCGG

Ayu (Plecoglossus altivelis)

(SEQ ID NO: 16)
atgctggccttcctggtactcatcctcttcctctaccatggaact
gtgtgtgtggagccagagagacacccacctgcaagggaggaagac
ctcccatggcagtcccatctttgctgtgacaccctgccccccctg
ccccccctgcccccccggctccccaggcccccccgtcccccccag
cctgggagatccagagaggagctctctgattcaaagaacgtccac
agctcctgcatctacttcaactcctccagcatggcatctcctcca
ccccccacagagagtcaagtgtgtttggatatccagtgtgtaatt
gacgcgagcggagagaaagtaatttgccatctgaaacctctcgat
gattcttccaactccaccagcggcttcacagtaatcaccctgcag
cgctctgaacgtggcgaggagagagagcgggaggaggtcgcgaac
gccagcgacaccgtcggctgcgcggggacgccgcccttccggtgc
tccgtcgccgtcggcgccgtcgtcgcggcaaccgtgaacgtgtcg
ggagccgtggctcccccggtgctgctccgccttctacccgttaac
ggtaaaccagacccaccagtgaacgttacgcacatcctcacccca
gacggagacctcctactgagttggagccccgcccccctcagctcc
gccccccccggctccgcctacctaagccccgccccgctccgctac
gaggtccgctatgcccccagcgcctcccctgctgtttggaaggcc
ccagctccgccccctcgcccttcctccttcctggagcgactggag
ccaggcttacaacttcctgttggacttgagaagaggaacaacaac
aagatgaaggtccacccccccctgcccccccgtccccctgccctc
agcccccctgccccccgcccctcagccctcctgccccctgccctc
agcccccctgcctccccgcccctcagcccccgccccagcccccct
ggccctcattacgccagctacttcccagagagggtggcggtccga
cgcggggaagacgtgagcctccgctgcttgttcaacgaccctgct
gccaagtccagcacctccgtgtggttcctgaactggcaccctgtg
gaggccaccgtccaccaggtggtagaggtcagccccagggtcagc
caggtcagccccagggtcagccaggtcggcaccagggtcagccag
gtcagccccagggtcagccaggtcggccccagggtcagccagatc
accctgcggccccctgaggaacgactgcacagcacgctgcagtgc
taccagagcagcaacactcgctactccttcaaggttccctacgct
gacgtctacgtccacggagcgctcatcgatatccgctgccagacc
aacggggtcatggacgccatgacctgcaggtggaacaacagccac
agactggaggagctcaccttcttctacaggtcggaagctgtgacc
tgtgacatcatggaggaggaaggagaggaggaggaggggagcgtg
gaagagggtcgcagtgagatcaacgggacaaagtgtccaatcagg
aaggaggaagggagtgggaggagctgtgttttccagccgctcaga
ctgacgtactgctacaagctgtggctgcagatccagtcagaggac
ggacccgtgaggtcacatcctgtctacgtcacgcccaccgaaaat
gtgaagcctaaccctccagcccaggtgcaggctcatgtctctccc
agcagaattctgaagcttgtctggcagcctcctagactgcccttg
tacgagctccagtaccaggtccgatacctccccgctgcaggtcgg
gccctcccagactggcaggaagtgggtccagtgagcgagccaaga
gcggagatccctgtacctgacgtgtgtggcgtgtacctggtccag
gtgcgctgtgtacccaccaatgggactggccactggagcgactgg
agcaagcctgtctactccacaacccccaaccgcagtgctccccat
catggtccagacttctggaggattctggaggaagattcatccttg
aacatcacctacgttactctgctcttcaagcatgtctctatggag
ccctcctcctgtgtagagggccttgcagttcagcaccaggtctct
gggggcgctgtggtcacggagctgatcggcctagttccttcttac
acgtttgaatggaaggaggaagtacacacggtgacggtgaaggcg
cagaatcgcttgggatcgtcaggtcacaacgtcaacatgaccttg
gaccgacacgtcaaacgtccgtgtttactctggttcagagcgtcc
ctggtcaacagcagctgtgtggagctatcctgggctctgctgccc
cagaacccgaccccctggtccctggtgctgcagtggtcaggagcc
accagggggcgccggccagcccacgacccgacctcggggaccagc
ctgaagtgggccagggtgccgttcactgaccgcgttttctacctg
cacggagaattctacggctctgaagagtatgagttcatcttgtac
cctatatttgcagacagggaaggcgagccaatgcatgctaaagcc
agcagagggccccctgctgactacatgttgctgattatcatcacc
ttcctagccatcgtcctcatcatcactctcatgttgttccagaac
cacatgaagaagctgatgtggagaaaggttcctaaccccaaccgc
tgctcctgggcgcaaggaagagacttccggaaggcagagaccatg
gaccagctgttcagacacccagacggtttccctgcctggcctctc
ctcctggtgtctgagaccatctctgaggtggtgatcatggagaag
agcagccttccctctcctgctctctctccagctccggaccccgtc
cctgggcaaggccctcccagaggcctccctggccctcgcccctcc
tccacccccagcgaaggctctggctctgggttccccaggacatca
gagacctcggcccagtcgtcggtcgcgtacgctactgttctcctc
tccaacaagccaagcctcctccgcaagcaagatggaggtagcagc
tccagcgacgaggggaacttctccgccaacaattcggacatttct
ggctccttccctgaggggttgtgggggctggacaccctgcggacc
ggagggcctgacaccagacattcacgctctttcaactccgtggag
gagttctccgaaacatcggagcaggaggacgaggcgttggggggg
gagggagagggggagggagagggggagggggaggagaagagcctg
tactatctagatatgggctgtccgcaaagggaggaggaggaggaa
gaggaggaggaggaggaggaggaggaggggaggttggaagaaagg
aaaacactgcttctccaggaacgggaggcgttgtgggcggagcct
aactctccgcttgggcggggcgaccccaggttcgagtgcagggag
gcggagtcaggtgacatggttggaggcgtggctctttacctgcct
cagtttaggactggccccggcagacctggtacagagggaaggcca
gacagcgccctccagctgtga

Chub mackerel (Scomber japonicus)

SEQ ID NO: 17
ATGAACACAACAATGGTTTGGTCTGTAATGTTGTCAGTCCTGATG
CATATTTTCCTGGTGTCCCATGGTGCCCTGTGTTTGGAGCCCGAG
GAGGTTGCCTCTCTTCAGGCAGGTGCTCTGAACCTCCCATGGCAG
GATGAGCTGTGCTGTGATTCACCGTCAGCCCACCTCACTGTGAAG
GTGGACGGTGTGCACGCACTGGAGACAAACCACTCTGAATCAAGC
CTCCCACACTATGCTCACTGCAACTTCAGGAGCCTGACAGCCACA
TCACATCCTCACAAGCCCTCTGGTGGCACCTGTTTGGACATGCTG
TGCAGAATTGATGAAAAGTGGGAGAGTTTGACTTGTGATCTTCAG
TCTCACGACCCGCCTTCAGACACAATGAATACTGCTCTTATGGCA
ATTAGCTTACAGCATCTGCTCTCCAAGAAAAAGGGCTCAGAGGTG
AATTATGGGAATGACGCATCTTATAACCCTGTCGTCTGTGAAGCA
GAGGATTCCTTCATGTGCTCACTCCCACTTGACACTACGACAAGC
TTTGTTACCATGGTGACTGTCAGCATCGCTGAAGCTGTAGCTCCG
CCAATTCTGCTCAGAGTTCCTGCCCGACCTGTGAAACCCAGTCCT
CCGGTCAACCTTTCACATATGCAGACTATTGAGGCAGAACTGATT
TTACACTGGGACGACCCAGAAGACATTGATTCTGGTCTGCTGAGA
TATGAGGTCCGATACTCTTCTAACACCAATCATCCAGCCTGGCAG
GTGGTTTCTGTACCCGCAGAGCCCAGGATGCTTCTGGATTTGAAG
CCCAGACTGAACTACACTATCCAGGTTCGCTCCGGCTTGGATGAA
CCCCCACTGTGGAGTGACTGGAGTGAACCCTACCACATTTACCTG
GACACGGTGAGCTACATCCCTGAGAAAGTGGTGGCGCGCCCGGGA
GAGAACGTAACAGTTTATTGTGTATTCAACGACCACAGCATCAAT
GCCAGCACGGCCATGTGGATGCTCAACTTCCAACAGCCGCTTCAT
CGCAGCCAGTACCACCCAGTCAACCAGTGGGTCAGCCAGATCACA
GTGCGCCCTTCAGAGACTCGAATGTATGACCTGCTGCAGTGCACG
CAGGAGTGGACCATCCCCTACAGCCAGATCTATGTAGAAGGAGCA
TCCATTGATATAAAATGTGAAACCAACGGTGATATTGATGCTATG
GACTGCAGCTGGAAGAACACACAGTGGACTAAACCCAAATTCCTA
TCCAGGTGGGCTGATCTGCCATGTGATGTAATGGAGGAAAGGGAG
AGAGCAGGTGAGAGTGTGGGAGAGATGGGACCTGTTTGCCTCTCT
CAGAAATCCTGCACCATCCAGCCCCTGAGGATGAACTGCTACAAG
TTGTGGCTGGAGGTGCCATCCCGTCTGGGCCCTATCTGGTCCAAA
CCTGTCTACCTGTCACCCATAGATCATGTGAAACCACACACACCG
ACTAATGTGAGGGCAGTGAGCCGGAGCAGCGGGGTCCTGACGATC
ACATGGGAACCACCTTCTCTGCCAGTTGAGGGGCTCCAGTGTCAG
TTTCGTCTCCACTCACTGTCCACTGTCAGAGCTCAGCCAGAGTGG
AAGGTTCATAATCCAGTGCGGGTGCAATGGGCGGAGGTTGCGGTT
CCAGATATGTGCCGTGTGTATGTGGTGCAAGTGCGCTGCATGCAC
ACCAACGGCACCGGCTACTGGAGCGACTGGAGTGACTCCATTTAC
TCTACGCCACAAAATAGCAGAGCTCCTGATCGTGGCCCTGATTTC
TGGAGAGTTCTACAGGATGATCCATACAGAAACCAAACTAATGTT
ACTCTGCTATTTCAGCCTCTCCTGACATCAGGGCACTCCTACTGT
GTGGATGGATTTATAGTCCAGCACCAGTCCTCAAGTGGCACTGTG
ATAAGGGAGCGGATCGAGCTGGTGTCCTCCTACAGCTTTGAGTGG
AACCAGGAGGTCCAAACTGTCACCGTGGAAGCCTACAATAGTCTG
GGGAGCTCGGCTAACAACATCAATATGACACTGGGGAGACAGCCC
AAACGCCGCTCTGTGCGTGCGTTCCGTGCGCTGGTTATCAACAGC
ACCTGTGTGTCTCTGTCCTGGAGCCTGCTAGACAACAGCTCTGTC
CCTCTATTCATGGTGGTCCAGTGGTTGCCACAGAGGCAACAGGAC
CCTCATCATCACAAAGGCCTGAGTGGAGAACCATGGGTCAGAGTG
CCTTACACTGATCGTCCTGTCTATTTAAGAGGTGATTTCTTTGGC
TCTGAGGAGTATGGGTTCTATCTGTATCCTGTGTTTGTTGAAGGA
GAAGGGGAGCCAGCATACACTACAGCCACCAGAGGAGACCCTGCA
GCCTACATGATGCTGATGATCATCTCCTTCCTCTCCATCGTCCTG
TTTGTCACCCTGGTCCTCTCCCAAAACCAGATGAAAAAGTTTATG
TGGAAGGATGTGCCCAACCCAAACAAATGCTCCTGGGCTAAAGGA
CTGGACTTCAAAAAGGCTGACACGTTCGACCACCTTTTCCAACCG
CCAGAGGGCCTGCCAGCCCGGCCACTGCTGCTCCCCTCTGAGAAC
ATTTCCAAAGTCGTTATTATGGAAAAGACTGACCTCTCAGCGCTG
ACCCCGCTTGTGTCCCTAAATCCCGACACAGCAACTGCCTTAGCT
ATCTCCATTCCTTCAGGGTTTAACTCTGAGGTTGACCAAACCCGA
TCCATGGACAGTGAAGCGCTTCTGGGTGGAGCTCCCTCCTTAACC
CTTGATATGGATGCTTTAACCGCCTCAAGCCTGAGGACAGATGAG
TTACAGCTTGTCGACCTCCCAAGAACAGATCAGCTGCCAGGCACC
AATAACAACAGTGCACAGTCCTCAGTCACTTATGCCACTGTGCTG
CTCATTAATCCAAAGCAGGAGCAGCAGCCCATTCATCTCCACTAC
AAGGATGGTAGTGGCAGCAGCTCCAGTGATGAGGGCAATTTCTCT
GCTAACAACTCAGACATTTCAGGATCTTTCCCCGGTGGCCTGTGG
GAGCTGGAGAGCTGCCGAGGTGGGGAGATGGATGACCCGCGACGC
TCCTGCTCCTATAACTCCGTGGAGGAGCTTTCTGAAACATCTGAG
CAAGAAGATGAGGAGGAGGTGAGAGAAGGGAAACACCTGTATTAT
CTCGGTATGGACTATCCAGCGGAGGATGAGGAGAGTGACGAAGAA
GAGGAGCAGAGAGAGGAAGATACAAAAACTGAGCTGCTCACAAGT
GTTGTTTTGAACAGAGATGCCTGCTCTGTGGAGTCACACCCATTG
CTTGGCCCTGTGGACTCGAGTGATAACAGTGAGATGGATTCAGCA
TCAACACGTGGCTTTTCCCTGCTGTACATGCCTCAGTTCAGAACT
GCACCATGCACGAGGCAACTCACAGATTAA

### (SEQ ID NO: 17)

Hong Kong grouper (Epinephelus akaara)

(SEQ ID NO: 18)
ATGGACTCGGACAAAAGAAGAGCCGGACACAAAGCCAGGGGAAAC
TTCCAGGAGCGTTTGAGATTCACCCCAGACAAAGCCCAGCCAGGC
CATGCTCACGCTATGACTACTACAATGGTTCGGTCTGTTATACTG
ACAGCCCTGATGCACTTGTTCCTGGTACCCCATGGTGCTCAGTGT
TTGGAGCCTGAAAACGGAGCCTCTGACCATTCAGGTGCCCTGGAC
CTCCCGTGGCAGGATGAGCTGTGCTGTGACTCACCTTCAGCCCAC
CTCACTCTGGAGGGAGACGACATGCACGCACCGGAGACAAACCGC
TCTGAATCAAACCTCCCACAACATCCCCTCTGCAGCTTCAGGGGC
TCGACAAGCGAATCACATCCACGTGAGCCCTCTGGCGTGAAACCA
AGTCCTCCAGTCAACCTGTTACATAATCAGACCATTGAGGCAGAC
CTGATTTTACAATGGGACGACCCGTCAGACTCTGATACCAGTCCA
CTGAGATACGAAGTCCGATACTCTTCTAACACCACTCATCCACAG
TGGCAGGTGGTGTCTGCACCTGGAGAGCCCAGGTTGTCTCTGGAG
CTGAAGCCCAGACTGAACTACACCATCCAGGTTCGCTGCTCCGGC
CTGGAAAACCCGCCAGTGTGGAGCGACTGGAGCGAACCTTATCAC
ATCTACCTGGACACGGTGAGCTACATCCCTGAGAAAGTGGTGGCA
CGGCCGGGGGACAACGTCACGGTGTATTGTGTGTTCAATGACCAC
AGCATCAACGCCAGCACGGCCATGTGGATGCGCAACTTCCAACAG
CGGCTTCATAGCAGCCAGTATCACCCAGTTAACCAGTGGGTCAGC
CAGATCACAGTGCGTCCTTCAGAGTCTCGGATGTACGACCTCCTG
CAGTGCACTCAGGAGTGGACCATCCCGTACAGCCAGATCTATGTA
CAAGGAGCTTCCATAGATATTACATGCGAAACCAACGGTGACATC
GACGCGATGACCTGCAGCTGGAAGAGCACACAGTGGACAAGAATC
AAATTCAGATCCAGGTGGGCTGACCTGCAGTGCGACGCGATGGAG
GAAAGGGAGCGAGCGGGGGAGAAAGTGGGGGAGTTGGGGCCCTCG
TGCCTGCAGGTCCGATCCAAGCAGAAAACCTGCACCATCCAGCCT
CTCAGGATGAACTGCTACAAGCTGTGGCTGGAGGTCCCGTCCCGA
CTGGGCCCCATCAGGTCTAAACCCATCTACCTGTCCCCCATCGAT
CATGTGAAACCCCACTCACCCACTAATGTGAAGGCAGTGAGCAGA
AGCAGCGGGGTCCTGATGATCTGTGGGAGCCTCCGCTCTGCCAGT
CGAAGGGCTCCAGTGTCAGTTTCGGTACCACTCACCCTCCGCTGT
GAGAGCCCAGCCGGAGTGGAAGAGGATGTTCTGACATGGACTCAG
CTGATCCAGAGTCCAGTGCGGGTGCCATGGGCGGAGGTCTTAGTG
CCGGACATGTGCCGGGTGTATGTTGTACAAGTACGCTGCATGCAC
ACCAACGGCACCGGCCATTGGAGCGAATGGAGCGATTCGGTCTAC
TCCACACCTCAGAACAGCAGAGCTCCTGAGCGAGGCCCCGATTTC
TGGAGAGTCCTTCAAGATGATCCGTACAGAAACCAGACGAATGTC
ACTTTGCTGTTCGAGCAGCAGCATCTCCAACTATCAGGACGGTCT
TACTGCATAGATGGATTTATAGTGCAGTACCAGGCGTTGAGTGGC
TCTGTGACGAGGGAGCAGATCGAGCTGGCGTCCTCCTTCAGCTTT
GAGTGGAACCAGGTGCCCCAAACTGTGACGGTGGAGGCCTACAAT
AATCTGGGGAGCTCCGCCAACAACTTCAACATGACGCTGGAGAGA
CAGCCTAAACGCCGCAGTGTGCGCTCGCTCAGCGTGTTGGTTATC
AACAGCACCTGTGTGACTCTGTCCTGGACTCTGCTGGACAACAAC
TCTGTGCCTCTGTTCATGGTGGTCCAGTGGTCTCCACATAAGCAA
CAGGACTCAGATCATCAGAAAGGCCGGATTGGAGAAACATGGGCC
AGACTGCCCTACACCGACCATCCCATCTACCTGAAAGGTGATTTC
TTTGGCTCTGAGGAGTGTGGCTTCTACTTGTACCCTGTGTTTGCT
GATGGAGAAGGGGAGCCGGTGTATGCTATAGCTTCCAGAGGAGAC
CCTGCAGCCTACATGATGCTGATGATCATCTCTTTCCTCTCCATC
GTCCTGTTTGTCACCCTGATCCTCACCCAAAACCAAATGAAAAGG
TTTGTGTGGAAGGATGTGCCCAACCCCAACAAGTGCTCCTGGGCC
AAAGGACTAGACTTAAAAAAGGCTGACAACTTTGACCACCTGTTT
CAACCTGCAGAGAGCCTGTCAGCCTGGCCGCTGCTCCTGCCAGCT
GAGAACATTTCAAAAGTCGTCATAGTGGATGAAGCTGATCTCTCA
GCTCTGACTACGGCTTTAATCCAAGTCCCACTTGTGTCCCTGACT
CCTGACCCAGCCACTGCTTTATCTATCTCCCTTCCTCCAGGGTAT
GACTCGGAGGTCGACCAAGCCCAGCCCATGGAGAGTGAGGTGCTT
GTCAGTGGAGCTCCATCCCAGCTCATGGAGCAGCCGCCAATTCAT
CTCCACTACAAAGATGGCAGTGGCAGCAGCTCCAGTGACGAGGGC
AATTTCTCTGCCAACAACTCAGACATTTCTGGATCTTTCCCCGGC
GGCCTGTGGGAGCTGGAGAGCTGCCGTGGTGGAGAGATAGACGAC
CCTCGGCGCTCCTGCTCCTACAACTCTGTGGAGGAGCTTTCTGAA
ACATCAGAGCAAGAGGATGAGGAAGAGGAGGCGAGAGAAGAGAAG
GACTTATATTATCTAGGAATGGACTACCCAGCGGAGGATGAGGAG
AGCGAGGAAGACAAGGAGCAGATAGAAGAAGAGACAGATATTGAG
CTACTTAAAAATGTGGTTTTGAGCAGGGAGGACTGCTCTGTGGAG
TTGCACCCTTTGCTCAGCCCTGAGGACTCGAGCGAAGTTGCTGTT
AGCGTCAACACGTGGTTTTTCCTCGCTGTACCTGCCTCAGTTCAG
AACTGCTCCATGCACGAGGAAACTCACAGCTCAACCGCATGA

Honmoroko (Gnathopogon caerulescens)

(SEQ ID NO: 19)
ATGATGTATTTGTTTATCTTGCTTTCGCTTCTTGCGAATTTCATT
ACCGTTTCACAAGGTCTTGCTGCTCTGAATCCTTCGGATGGCAGG
GGAGTTTATGAAGACCTGAAGTGGAAGTCTCTGCTATGTTGTGAG
CTGCCCTTTGCTCAGACAGTTGACAGTGGTCTTTCAGAACACCGA
CCAGTAGAACCGTGTCAGCTGCTAAATGTTACAAAACCGGAATCC
TCTAGTAAATCACTTGCCTTTTCTGGAAACTGTTTGGACATCCTG
TGCTGGCTTGAAGGTGAACGGACAAATTTGATTTGTAATGCAAAG
AGCCACGGAGAAGCAGCCACTGCCAGTCTGTTCACTGTTAGTCCT
CAACAATTAGTTCTACAGATGGATATACTTTCAGAAAAAAACCAT
AGTGCTCAGTGTGCTGGGGAAGATACTACCACGTGCTCCGTTTCT
CTTCATGGCAATGATGCAACTGTCTCGTTGACCATCAGTATATCT
GTGAATGGGACTACTGCACTGTCACCGGAGATGCAAATCAGCACA
TATCATCTAAGAAGACCAGACCCACCTGTCAATCTGCATTACAAT
GTGACGACAGAAGACGAAGTCATTTTCAGATGGAGTAGCACACAA
CCTGACAGTAATGACATAAACTATGAGATCCGATACTCTTCCAAT
TCCTCGCTTCAGCAGTGGGAGGTGGTGAAGGTCAGAGGTCATTCA
TGGGTACCTCTGAATGAGCTCAGTTTTGGGATCAGATACACAGTC
CAAGTGCGCTGCCAAAACTACTTCCATTACTGGAGTGAATGGAGC
CAACCTTTCTATACACTAGACGTCAGTTACATCCCTGCTGAAGTC
TCCACAACGCCTGGGTCAGAGGTAACGGTTTATGCCGTCTTCCAC
AACCGCAGCTGGAGTGCGAGCAAAGCTGTGTGGATGCTAAACGGG
CATGAGAAGATTCCGGAAAGCCAGTACAGGGTAATCAACGAGCAA
GTTCGTGCTGTCACTATAAGGGTAGATGAACCCAGGTTTGATTCT
CTGATGTGCTGCCACTTGGGAGGAGAGCGGGTCAAATGCCCCATC
GCCTATGGCAAAATATACACCGAAGGGAGTTTTAATGCAGATATT
ACCTGCGAGAGCAAGATTTCAGAGGTGGACACCACTCTGGATTGT
GAGTGGAATAAAAGCGCTTGGGCACTGGTCAGACTCCTTTACAGA
CAATATATATCAATATGTGAAACCATATCAAAGATGCAGGGCATT
GAAGAAGCTGAAGAAAACATGCCTTTGGTAAAGGAGTACCAAAGT
GGAGCAGGAGACTACAGAAAATACTCTTTAAGCAACCTTAGCCTG
TACTCTTGTTACGAAATCTGGTTGGAAGTGGAAGGAGGGCGTGGC
AAAGTGAGATCATTTCCTGTCTATGTTTCACCCATTGACCATGTG
AAACCATCTCCTCCCTCAGTCCTTGAAGCGATCACTTTGCCGAAC
AAAACCTTAAGCGTAAAGTGGAAATATCCCGATTTACTTGTATAC
AAAATGCAGTATGAGCTGCGCTTTGTGGCTTTGCGTGGGATGGCA
AATACGCAATGGAAGGTCATCGGTCCTCTATTGGAAGCACAGGCA
GAGATTCCGCTTGAAGATTCCTGTGTTCAGTTTAATGTAGACGTT
CGCTGTAAAAGACTGAATGGCTCTGGATACTGGAGCAAATGGAGC
GGGAGTCACACTTCCATTGTTTACAACAGGAAAGCTCCTGAAATT
GGACCAGACTTCTGGCGCATTATACAAGAGGATCCTGTTAGGAGC
GTGACAAATGTCACACTGATCTTTAAGCAGCCTATCCTAGCAGGA
GATCCATATAGTTGTGTGGAGGGTCTCAAGATTAAGCACCAGGCC
TCAGGTGAAGCCGAGTGGTCAAAAGAAACGGCCTTGATTCAGTTT
CACTCATTCCAGTGGAAGAAGGAAGCACACACAGTCACCGTAATG
TCTCGCAACGCTTTGGGCTTCTCTACACGGAATAGCAACATGACG
CTGTTACATCAACCCAAACGACGATGTGTACGGTCATTCAGCGCA
GTTGCAAATGCGAGCTGTGTGCATCTCTCCTGGAGCCTGTTACCT
GATCAACTGGTGCCTCAATCGTTTGTCATCGAGTGGTTAGACCTG
AACAAAGATCCTGAACAGGACGGGTCTCTAACGGAGAGGCTACAG
TGGGTCAGAGTTCATTCCGCATCCAGGGATATCTCTCTTTGCCGT
CGTTTTTATGGCTCGGAGGAGTTCACATTGTATCCAGTGTTTGTG
GATGGTGAGGGGGAGCCGGTTCGATACACAGTTACTAGGAGCGAC
TCTGCTGCCTACATACTGCTACTGATCATTGCGTTCCTGTCTGTG
GTGCTGTTTGTCACACTCATGATGTCACAAAACCAGATGAAAAAA
CTTGTGTGGAAGGATGTTCCAAATCCCAACAACTGCTCCTGGGCC
AAAGGAATGGACTTCAGGCAGATTGACACCATGGAGAACCTGTTC
CCTCACTCAGAAGGTCTCACGGCCTGTCCACTGCTGCTGGTCTCC
GAGAGCATCTGCGAGGTGGAAATCGAGAAATGTCATCCTCTCAAC
CTTGAACATGGAAAAGACAATGAAGTACTCCTTTATAACTCGGAA
GACAAGACAACCACCGACTCTGCCCGTCTGGGAGACTCCTCTGAA
CCTTTATCACTGGAGGCTTCCACTGCTGCTGCTACCCCAGAAACA
TCAGGCCAGTCCTCGGTGACATACTCGACCGTCCTCCTCGCCGAT
CAGCCCGCTCTCCTTCGAAAGCAGCAGGAGAGTCTGAGCAGCTCC
AGCGACGAAGGCAACTTCTCTGCTAACAACTCAGACATTTCCGGC
TCCTTCCCTGGAGGACTCTGGGACCTGGAGAACCACGTGTGCTCT
GACAGCACCAATCCTCGCCATTCCAGCTCCTACAACTCCGTGGAA
GAGTTCTCGGAAACATCAGAACAAGATTACGAAGCATCGGAAAGC
ACTGGTGTAGCCAAAGACCTCTACTACCTTGATATGAATGAAGAG
GAGAAGCAGGACAAAGAAGAGGATGAGGAAGCTCAGGATGGACAG
GATAAGAATGAAATGGTTATGAGGGTGGATGCCAGGCCTCTTCTG
GAAGGCAAAAATTCCACAGACGTCGATTCCAATAATGTATCTCAC
AGCGTTCCGCTTTATCTGCCTCAGTTTCGAACTGAATGCATCAAT
CCACCATGA

### SEQUENCE LISTING

1. A method for promoting sexual maturation of fish, the method comprising:
a step of suppressing functional expression of at least one of a leptin receptor and a leptin of the fish.

2. The method according to claim 1, wherein
the functional expression is suppressed by introducing a loss-of-function mutation in a gene of at least one of the leptin receptor and the leptin.

3. The method according to claim 2, wherein
the loss-of-function mutation is introduced by at least one genome editing system selected from a clustered regularly interspaced short palindromic repeats/CRISPR associated protein 9 (CRISPR/Cas9) system, a CompoZr Zinc-finger nuclease (ZFN) system, and a TAL effector nuclease (TALEN) system.

4. The method according to claim 1, wherein
the functional expression is suppressed by suppressing expression of a gene of at least one of the leptin receptor and the leptin.

5. The method according to claim 4, wherein
the expression of the gene is suppressed by at least one type selected from a group including at least one type of an RNA molecule selected from a group including a SiRNA, a shRNA, and a miRNA targeting mRNA of at least one of the leptin receptor and the leptin, or a vector capable of expressing such an RNA molecule.

6. The method according to claim 1, wherein
the fish is a female individual.
